(19) United States
(12) Patent Application Publication
(10) Pub. No.: US 2026/0083351 A1
(43) Pub. Date: Mar. 26, 2026

(54) MEASUREMENT OF LUNG FUNCTIONS BY PULMONARY GAS EXCHANGE

(71) Applicant: The Regents of the University of California, Oakland, CA (US)

(72) Inventors: Peter D Wagner, La Jolla, CA (US); Atul Malhotra, La Jolla, CA (US); Gordon Kim Prisk, La Jolla, CA (US)

(21) Appl. No.: 19/110,989

(22) PCT Filed: Sep. 13, 2023
(86) PCT No.: PCT/US2023/074122

§ 371 Date: Mar. 12, 2025

Related U.S. Application Data
(60) Provisional application No. 63/375,527, filed on Sep. 13, 2022.

(51) Int. Cl. A61B 5/083

(52) U.S. Cl. CPC A61B 5/0836; A61B 5/0833

(57) ABSTRACT

Provided are methods and systems for measuring lung function, such as shunt and deadspace, using pulmonary gas exchange in management and treatment of pulmonary diseases including COVID-19.

### CROSS-REFERENCE TO RELATED APPLICATION(S)

This application claims the benefit of U.S. Provisional Patent Application No. 63/375,527, filed on Sep. 13, 2022, the contents of which is incorporated by reference in their entirety.

### BACKGROUND

For oxygen (O₂) and carbon dioxide (CO₂) exchange to occur in the lung, inspired gas (ventilation) and blood flow (perfusion) must be brought together in the alveoli. Regions of the lung that are not ventilated cannot exchange gas, and such regions are termed “shunt.” Regions of the lung that are not perfused also cannot exchange gas and are termed “deadspace”. There are two sources of deadspace. The first is anatomic deadspace—the conducting airways that transport gas from the mouth to the alveoli but do not partake in the exchange of O₂ and CO₂, which are present in all people. The second is alveolar deadspace—alveoli that would under normal circumstances exchange O₂ and CO₂, but when blood flow is interrupted, gas exchange does not take place. The sum of these two sources is the total deadspace. Anatomical deadspace is easily measured by established methods. However, alveolar deadspace is difficult to measure, especially as anatomic deadspace usually makes up the majority of the total deadspace.

The present technology proposes a generally applicable bedside gas exchange approach to identifying areas of high ventilation perfusion ratio (high \dot{V}_{A}/\dot{O}) that occur as a result of pulmonary vascular obstruction (PVO) or lung blood flow restriction to areas of the lung that result from pulmonary emboli, micro-emboli, or high positive ventilation pressures occurring alone or in combination with areas of absent ventilation or reduced ventilation (low \dot{V}_{A}/\dot{Q}) that result from pneumonia, lung collapse, airways obstruction and other syndromes. This approach addresses both the theoretical and practical aspects of solving this problem.

### BRIEF DESCRIPTION OF THE DRAWINGS

FIG. 1 shows a two-compartment model of the lungs with total alveolar ventilation (\dot{V}A) and blood flow (O) split between both compartments (\dot{V}A₁ and \dot{V}A₂; \dot{Q}₁ and \dot{Q}₂) resulting in corresponding \dot{V}_{A}/\dot{Q} ratios (\dot{V}A₁/\dot{Q}₁ and \dot{V}A₂/\dot{Q}₂). The different \dot{V}_{A}/\dot{Q}ratios then dictate corresponding values of alveolar PO₂ and PCO₂ in each compartment as shown. Arrows indicate the mixing of exhaled gas from the two compartments and mixing of compartmental blood flow from the compartments to form the mixed exhaled gas and mixed arterial blood.

FIGS. 2A-2B show the relative arterial-alveolar partial pressure differences arising from shunt and deadspace and from low or high \dot{V}_{A}/\dot{Q}ratio regions as a function of gas partition coefficient (A, abscissa). The ordinate cannot be less than zero nor greater than 1.0. The vertical dashed lines indicate the average values of the O₂ and CO₂ dissociation curves slopes expressed as partition coefficients (see text). FIG. 2A shows that the greatest impediment to gas exchange in the examples of low \dot{V}_{A}/\dot{Q}regions and shunt occurs for gases of λ=0.17 and 0, respectively. The O₂-equivalent gas has a more than threefold higher arterial-alveolar difference than the CO₂-equivalent gas. FIG. 2B shows that for the high \dot{V}_{A}/\dot{Q} and deadspace models, the most affected gases have λ=5 and infinity, respectively. The CO₂-equivalent gas has an arterial-alveolar difference about 50% greater than that of the O₂-equivalent gas. Whether the lungs have low and high \dot{V}_{A}/\dot{Q}regions or zero and infinitely high \dot{V}_{A}/\dot{Q} regions is seen to not be important for O₂ or CO₂ in each case (comparing lower two circles with top circle). O represents blood flow; \dot{V}A represents alveolar ventilation.

FIGS. 3A-3B compare arterial-mixed alveolar PCO₂ differences for three models (shunt only, deadspace only, and both). FIG. 3A shows the arterial-alveolar partial pressure differences for CO₂ (ordinate) plotted against the alveolar-arterial partial pressure differences for O₂ (abscissa). The points represent differences due to shunt alone (from 0% to 50% of the cardiac output); differences due to deadspace alone (from 0% to 50% of the ventilation); and differences due to shunt plus deadspace (each from 0% to 50%). It is evident that although both O₂ and CO₂ are affected by shunt, by deadspace, and by their combination, the relationships between O₂ and CO₂ for each type of abnormality are very different. A normal young subject, with atypical alveolar-arterial PO₂ difference of 10 mmHg and arterial-alveolar PCO₂ difference of 1 mmHg is shown by the yellow solid circle. FIG. 3B shows the plots of FIG. 3A extended into a complete grid showing alveolar arterial differences for O₂ and CO₂ as a function of the range of combinations of shunt and deadspace, each from 0% to 50%. This grid can be used for any measured pair of alveolar-arterial differences for O₂ and CO₂ to estimate the percentage of pulmonary blood flow perfusing unventilated regions (shunt) and percentage of alveolar ventilation associated with unperfused regions (deadspace). aAPCO₂ represents arterial-alveolar PCO₂ difference; AaPO₂, alveolar-arterial PO₂ difference.

FIG. 4 shows an expiration (left), an inspiration (middle), and another expiration (right) for a normal subject (curved tracings). For this conceptual example, the second expiration is a reproduction of the first. Tidal volume is a little less than 1,000 mL. Top panel shows PCO₂ and bottom panel shows P_{O}_{₂}. Vertical lines separate expirations from inspirations. The regression lines (black) for the alveolar plateau for each gas are linearly projected to the start of expiration and connected by the presumed linear return pathway for the inspiratory portion, highlighting the oscillation in alveolar gas levels between inspiration and expiration, marked by the horizontal lines drawn at the high and low points on the regression lines. The inspiratory segment shows inspired PO₂ and PCO₂. The expirations show initial exhalation of previously inspired conducting airway gas (first ˜100 mL), the transition toward alveolar gas (next ˜200 mL) and the linear, sloping, alveolar plateau (remainder of breath). The solid black circle indicates the mean alveolar P_{O}_{₂} and P_{CO}_{₂}, which are the values at the midpoint of expiration.

FIG. 5A-5C shows expired gas tracings from three subjects (FIG. 5A: normal subject; FIGS. 5B and 5C: two patients with COVID-19) analyzed by the proposed methodology. For both O₂ and CO₂, a single expiration is shown (curved line) with the least squares best-fit line to the latter portion of the exhalation (straight line). Solid black circles indicate the measured arterial PO₂ and PCO₂ and mean alveolar PO₂ and PCO₂. Alveolar arterial differences, shunt, and deadspace are indicated on the figure for each subject. The normal subject has minimal shunt and deadspace whereas both patients with COVID-19 have substantial shunt. However, only patient in FIG. 5C has a large alveolar deadspace in which the arterial-alveolar PCO₂ difference cannot be explained by the coexisting shunt.

FIG. 6 shows expired gas tracings from one patient: exhaled partial pressure of oxygen (P_{O}_{₂}; top black line, top symbols and lines) and carbon dioxide (P_{CO}_{₂}; bottom black line, lower symbols and lines). The second dotted line indicates the beginning of alveolar emptying (phase III), sloping dashed lines indicate the linear regression fits to phase III. Alveolar partial pressures (P_{ACO}_{₂} and P_{AO}_{₂}) were measured from the mid-volume of the linear regression fit to phase III (˜225 mL in this example). Filled symbols indicate mean alveolar values; open symbols indicate contemporaneous arterial values; arterial-alveolar differences are indicated by the solid vertical lines connecting the symbols.

FIGS. 7A-7B show alveolar-arterial partial pressure differences and alveolar deadspace in acute COVID-19 patients. FIG. 7A shows alveolar-arterial partial pressure differences for oxygen (P_{A-aO}_{₂}) and carbon dioxide (P_{a-ACO}_{₂}). FIG. 7B shows Intrapulmonary shunt and alveolar deadspace. Acute COVID-19 patients (n=30) and healthy subjects (n=13). Dotted lines in (FIG. 7B) show 95% upper limits for normal (52).

FIGS. 8A-8D show individual (symbols and lines) and grouped data (box-and-whisker plots showing mean, interquartile range and range) for 17 patients during acute COVID-19 infection and again in early recovery, together with 13 healthy individuals: alveolar-arterial partial pressure differences for (FIG. 8A) oxygen (P_{A-aO}_{₂}) and (FIG. 8B) carbon dioxide (P_{a-ACO}_{₂}), and (FIG. 8C) shunt and (FIG. 8D) alveolar deadspace. Horizontal dotted lines in (FIG. 8C) and (FIG. 8D) show the 95% upper confidence intervals for normal. *: p<0.05; **: p<0.0001.

FIG. 9 shows shunt and deadspace trajectories from acute COVID-19 illness to recovery. Dashed lines connect paired acute and recovery data (n=17). Vertical and horizontal dotted lines show the 95% upper confidence limits of normal.

FIGS. 10A-10D show individual data for 59 post COVID-19 patients NIH 1-2 (44 patients with low-medium severity, open circles) and NIH 3-5 (15 patients, enclosed squares) for (FIG. 10A) arterial PO₂ (FIG. 10B) arterial PCO₂, (FIG. 10C) alveolar PO₂ and (FIG. 10D) alveolar CO₂. **p<0.005 compared to low-medium severity. Black horizontal line represents median value.

FIGS. 11A-11D show individual data for 59 post COVID-19 patients NIH 1-2 (44 patients, open symbols) and NIH 3-5 (15 patients, closed squares showing (FIG. 11A) AaPO₂ mmHg (FIG. 11B) aAPCO₂ mmHg (FIG. 11C) shunt (FIG. 11D) alveolar deadspace. **p<0.008 compared to low-medium severity. Black horizontal line represent median values, dotted lines represent 95% confidence levels forshunt (5%), and alveolar deadspace (10%) from healthy subjects (73).

FIGS. 12A-12D show individual data from 7 healthy subjects (closed black triangles; FIG. 12A, FIG. 12C) and for 59 post COVID-19 patients (FIG. 12B, FIG. 12D) NIH severity 1-2 (44 patients; open circles) and NIH severity 3-5 (15 patients, closed squares) showing: (FIG. 12A, FIG. 12B) measured AaPO2 mmHg and aAPCO₂ mmHg, and (FIG. 12C, FIG. 12D) calculated shunt and alveolar deadspace values. Dotted lines represent 95% confidence levels for shunt (5%), and alveolar deadspace (10%) from healthy subjects (46). The healthy subjects' data lie within, or close to, the 95% confidence levels, indicating technical adequacy of the AaPO₂ and aAPCO₂ measurements.

FIGS. 13A-13B show individual data for 59 subjects for (FIG. 13A) Bohr-Enghoff deadspace (calculated as ((PaCO2−PECO2)/PaCO2)×100) plotted against measured alveolar deadspace as described in the text and (FIG. 13B) AaPO2 from the standard alveolar gas equation, where PAO2=PIO2−PaCO2/R+PaCO₂×FIO2×(1−R)/R plotted against AaPO2 using measured PAO2 as described in the text. Lines of identity are narrow, linear regression lines in bold. Note that Bohr-Enghoff deadspace measurements are much greater than the measured alveolar deadspace measurement and AaO₂ gradient calculation underestimates the true AaO2 gradient.

FIGS. 14A-14B show individual data for 59 post COVID-19 patients for (FIG. 14A) shunt and (FIG. 14B) alveolar deadspace plotted against time since acute SARS-CoV2 infection for post COVID-19 patients with low-medium severity (44 patients, NIH-1 and 2, open circles) and severe-critical severity (15 patients, NIH-3-5, closed squares). Dotted lines represent 95% confidence levels for shunt (5%), and alveolar deadspace (10%) from healthy subjects (73).

FIGS. 15A-15D show individual data for 59 post-COVID patients NIH severity 1-2 (44 patients, open circles), and NIH severity 3-5 (15 patients, closed squares) and methodological controls (7 subjects, black triangles) for relative ventilation plotted against (FIG. 15A) PaO₂, (FIG. 15B) Intrapulmonary Shunt (FIG. 15C) PaCO₂ and (FIG. 15D) Alveolar Deadspace. For comparison in A, historical data from healthy controls for Wagner et al (closed black circles (81)), Torre-Bueno et al (open black circles (83)) and Hammond et al (closed black squares (82)) are also plotted. Vertical line (FIG. 15B) 5% shunt (FIG. 15C) PaCO₂=40 (FIG. 15D) 10% Alveolar deadspace. Curved lines in C represents V_{A}rel=40/PₐCO₂, V_{A}rel=40/(PaCO₂−5), and V_{A}rel=40/(PaCO₂−10) if P_{A}CO₂=PaCO₂, and horizonal line is at V_{A}rel=1.0.

FIG. 16 shows PaO2 plotted against 40/alveolar partial pressure of CO2 (PaCO2), indicating alveolar ventilation (Va) relative to that which would be present in the same patient had PaCO2 been normal at 40 mm Hg (n=30; solid circles). In no patient was relative Va less than 1.0 or less than that calculated for healthy young adult subjects from Wagner and colleagues (107) (open circles, dashed lines representing 7 males and 1 female; mean±SD age, 29.8±6.1 yr), Hammond and colleagues (108) (open triangles, dashed lines representing 10 males; mean±SD age, 22.0±1.2 yr), and Torre-Bueno and colleagues (109) (open squares, dashed lines representing 9 males; mean±SD age, 26.0±6.0 yr). PaCO2 for historical control subjects was calculated using measured PaCO2, and the multiple inert gas elimination technique was used to measure \dot{V}a/\dot{Q} inequality to estimate the arterial-alveolar difference. Vertical dotted line represents PaO2=50 mm Hg.

### DETAILED DESCRIPTION

Provided herein are methods and systems for measuring lung function, such as shunt and deadspace, using pulmonary gas exchange to identify PVO occurring alone or in combination with pneumonia in lung disease treatment including COVID-19. The present technology is based on knowing that poorly (or non) ventilated regions (low \dot{V}_{A}/\dot{Q} areas) affect O₂ more than CO₂, whereas poorly (or non) perfused regions (high \dot{V}_{A}/\dot{Q}areas) affect CO₂ more than O₂. Exhaled O₂ and CO₂ concentrations at the mouth are measured over several breaths to determine mean alveolar P_{O}_{₂} and P_{CO}_{₂}. A single arterial blood sample is taken over several of these breaths for measurement of arterial P_{O}_{₂} and P_{CO}_{₂}. The resulting alveolar-arterial PO₂ and P_{CO}_{₂} differences (AaP_{O}_{₂}, aAP_{CO}_{₂}) are converted to corresponding physiological shunt and alveolar deadspace values using the Riley and Cournand 3-compartment model. To do so, a computer model of pulmonary gas exchange developed in the late 1960s by John West was modified to determine, in a novel manner, the amount of pneumonia and PVO that must be present in the lung to explain the observed AaP_{O}_{₂}, aAP_{CO}_{₂}. More specifically, the quantitative description of the consequences of areas of both high \dot{V}_{A}/\dot{Q}regions (termed alveolar deadspace herein) and low \dot{V}_{A}/\dot{Q} regions (termed shunt herein) in the lung on arterial blood gases was developed by West in the form of a computer algorithm. West's model answers what is called the “forward” problem: How do areas of high and/or low \dot{V}_{A}/\dot{Q}change arterial P_{O}_{₂} and arterial P_{CO}_{₂} and the alveolar-arterial differences? This algorithm was modified to run in reverse, i.e., to calculate the amount of shunt and alveolar deadspace that must be present in the lung to result in the measured values of the AaP_{O}_{₂} and aAP_{CO}_{₂}.

The present technology offers at least three advantages. First is the important (and usually ignored) fact that the presence of shunt alters the measurement of deadspace, and that the presence of deadspace affects the measurement of shunt. The present technology determines both simultaneously and in doing so, deals with this important interaction, an interaction which can result in incorrect estimates of shunt and deadspace by existing methods.

Second, mean alveolar P_{O}_{₂} and P_{CO}_{₂} was determined not by measuring the values at the end of a breath or in mixed exhaled gas (as is conventionally done), but by using the values from the mid-point of the expiration, and in doing so values were obtained for gas in the lung that are the average value over each breath, thus properly corresponding to the measurement of the arterial blood gases, which are also the average over several breaths. The values at the end of a breath are often used as estimates of alveolar P_{O}_{₂} and P_{CO}_{₂}, but these are usually the highest (for CO₂) and lowest (for O₂) over the breath cycle and are thus not representative of the average values. Furthermore, the approach of the present technology excludes the anatomic deadspace, allowing focus on the alveolar deadspace present in the gas exchange region of the lung, which has been hard to measure because the anatomic deadspace usually makes up the majority of the total deadspace.

Third, the present technology builds on the work by John West, who quantitatively showed how to calculate the consequence of shunt and deadspace on pulmonary gas exchange. The present technology modifies this work to ascertain the amount of simultaneously present shunt and deadspace that must exist in the lung to explain the measurements of pulmonary gas exchange seen in that lung. Put differently, the present technology modifies John West's approach for the “forward problem” and uses it as the basis of a solution of the “inverse problem”.

On the basis of physiological principles underlying gas exchange, the present technology proposes that simultaneously assessing O₂ and CO₂ exchange across the lungs will provide a clinically feasible approach to inferring the presence of diffuse pulmonary vascular obstruction in patients, both those breathing spontaneously and those on ventilators. Such obstruction may be due to massive clot (pulmonary embolus) or due to microvascular clot, as has been suggested to be prevalent in severe COVID-19 disease, or may be due to other causes of regions of high \dot{V}_{A}/\dot{Q}, including high positive pressures in severely ill ventilated patients. Thus, while the present technology was initially stimulated by the COVID-19 pandemic, it has potential wide-ranging applicability in other lung diseases.

If pneumonia, giving rise to alveoli of reduced \dot{V}_{A}/\dot{Q}ratio, is the primary consequence of COVID-19 or other disease, O₂ exchange will be affected more than that of CO₂. In contrast, with vascular obstruction creating alveoli with elevated \dot{V}_{A}/\dot{Q}ratios, CO₂ exchange will be relatively more affected. These consequences-singularly and in combination—can be identified by measuring partial pressures of O₂ and CO₂ in the alveolar gas and in the arterial blood and determining the alveolar-arterial gas tension differences for each gas. These measured values can be converted into values for physiological shunt and alveolar deadspace. Although the stimulus for developing this methodology was COVID-19, it is generally applicable to patients with pulmonary disease of any cause.

The present technology provides quantitative measures of simultaneously present shunt and deadspace and has potential to alter patient care. While the estimation of shunt in acutely ill patients, for example, patients in intensive care units (ICU), is well established, measurement of deadspace is rarely performed largely due to the difficulty of making the measurement. As such, it is often ignored. Having access to both parameters on the basis of a single set of measurements would alleviate this gap. At present, the focus on gas exchange defects is usually on shunt and the resulting arterial hypoxemia. Having quantitative values for both shunt and deadspace would potentially open up a number of lines of improvement in medical care including the following: (1) Diagnosis of the presence of alveolar deadspace in patients with acute COVID-19 or pneumonias of other origin. In cases in which alveolar deadspace is elevated, anti-coagulation therapy might be indicated, however such therapy might be considered inappropriate in the absence of elevated alveolar deadspace due to the potential for unwanted side-effects. (2) Determination of whether high alveolar deadspace persists, following recovery from acute disease (COVID-19 or other disease), again allowing for the administration of appropriate pharmacological intervention only if indicated. (3) Monitoring the recovery from pulmonary embolism by allowing quantitative measurement of the resolution of alveolar deadspace, allowing for the discontinuation of some therapy to be based on measured parameters of recovery. (4) Better insights into the presence of alveolar deadspace in numerous other diseases, something that is generally unrecognized largely due to the inability to quantify it. (5) Using changes in alveolar deadspace as a warning sign of possible impending lung damage from mechanical ventilators when high inflation pressures are used.

The present technology may have two possible “modes” of operation: (1) As a physical device containing conventional O₂ and CO₂ gas analyzers and an air flow meter. These components will make the measurements of alveolar partial pressures. Then, the arterial blood gas results measured by a separate device (clinical blood gas analyzer) could be entered from a keyboard. The device, containing a dedicated programmed microprocessor, would then perform the necessary calculations and report the resulting shunt and deadspace values. In this manner the device may be similar to (albeit more complicated than) the Alveolar Gas Meter of John West which is marketed though MediPines. (2) The invention could alternatively be implemented in a Software as a Medical Device (SMD) manner. In this case the user would be responsible for the measurement of the necessary parameters in exhaled gas and arterial blood, and the software would determine the values for shunt and deadspace from these data.

In some embodiments, the present technology includes a method of estimating pulmonary shunt and/or deadspace in a subject. In some embodiments, the subject is suffering from a pulmonary disease. In some embodiments, the pulmonary disease is asthma, pneumothorax, atelectasis, bronchitis, chronic obstructive pulmonary disease (COPD), lung cancer, lung infection, pulmonary edema, empyema, lung abscess, tuberculosis, cystic fibrosis, pulmonary embolus, and/or another pulmonary disease known to one of skill in the art. In some embodiments, the pulmonary disease is lung infection. In some embodiments, the lung infection is caused by bacteria, virus, or fungi. In some embodiments, the lung infection is caused by Streptococcus pneumoniae, Haemophilus species, Staphylococcus aureus, Mycobacterium tuberculosis, or another bacterium known to cause lung infection. In some embodiments, the lung infection is caused by influenza virus, rhinovirus/enterovirus, parainfluenza, respiratory syncytial virus, adenovirus, coronavirus, or another virus known to cause lung infection. In some embodiments, the lung infection is caused by Histoplasma, Coccidioides or Blastomyces, or another fungus known to cause lung infection. In some embodiments, the lung infection is caused by COVID-19.

In some embodiments, the method of estimating pulmonary shunt and/or deadspace comprises (a) measuring oxygen (O₂) and carbon dioxide (CO₂) concentrations over multiple breaths of the subject; (b) determining a mean alveolar O₂ pressure (AP_{O}_{₂}) and a mean alveolar CO₂ pressure (AP_{CO}_{₂}) based on the measurement of step (a); (c) obtaining a single arterial blood sample from the subject; (d) determining an arterial O₂ pressure (aP_{O}_{₂}) and an arterial CO₂ pressure (aP_{CO}_{₂}) from the arterial blood sample of step (c); (e) calculating an alveolar-arterial O₂ pressure difference (AaP_{O}_{₂}) and an arterial-alveolar CO₂ pressure difference (aAP_{CO}_{₂}); and (f) estimating a shunt value and/or a deadspace value using the Riley and Cournand 3-compartment model based on the AaP_{O}_{₂} and aAP_{CO}_{₂} of step (e).

Measuring oxygen (O₂) and carbon dioxide (CO₂) concentrations over multiple breaths of the subject may be performed using any technique known to one of skill in the art. For example, measuring oxygen and carbon dioxide may be performed using techniques routinely used in cardiopulmonary exercise testing (CPET). In some embodiments, the subjects breath rate may be measured using a flow meter. Measuring oxygen (O₂) and carbon dioxide (CO₂) concentrations over multiple breaths of the subject includes measuring exhaled oxygen and carbon dioxide.

In some embodiments, oxygen and carbon dioxide concentrations may be measured over multiple breaths of the subject. In some embodiments, oxygen and carbon dioxide concentrations may be measured over 2-10 breaths of the subject. In some embodiments, oxygen and carbon dioxide concentrations may be measured over multiple breaths while the subject's breathing is in a steady state. As used herein, a subject's breathing is in a “steady state” when the multiple breaths are substantially the same duration. In some embodiments, a metronome is used to ensure that the subject is breathing in a steady state. In some embodiments, the tidal volume of the subject's breaths is sufficient to produce an alveolar plateau.

In some embodiments, the oxygen and carbon dioxide are measured at the point halfway through exhalation. In some embodiments, mean alveolar oxygen and carbon dioxide levels are determined as the average values of oxygen and carbon dioxide at the point halfway through exhalation. In some embodiments, mean alveolar oxygen and carbon dioxide levels are determined as the mean values of the oxygen and carbon dioxide levels at the point halfway through exhalation averaged over the entire respiratory cycle. In some embodiments, an oxygen analyzer and/or a carbon dioxide analyzer is used to measure the oxygen and carbon dioxide in the subject's breath.

Determining a mean alveolar O₂ pressure (APO₂) and a mean alveolar CO₂ pressure may be performed using methods known to one of ordinary skill in the art.

Obtaining a single arterial blood sample from the subject may be performed using methods known to one of skill in the art. In some embodiments, the single arterial blood sample is obtained while the subject is breathing in a steady state. In some embodiments, the single arterial blood sample is obtained while the oxygen and carbon dioxide levels are being measured from the breath of the subject. In some embodiments, the single arterial blood sample is obtained over at least 2-4 steady state breaths of the subject.

Determining an arterial O₂ pressure (aP_{O}_{₂}) and an arterial CO₂ pressure (aP_{CO}_{₂}) from the arterial blood sample may be performed using methods known to one of skill in the art.

Calculating an alveolar-arterial O₂ pressure difference (AaP_{O}_{₂}) may be performed by subtracting arterial O₂ partial pressure from the alveolar O₂ partial pressure. Calculating an arterial-alveolar CO₂ pressure difference (aAP_{CO}_{₂}) may be performed by subtracting the alveolar CO₂ partial pressure from the arterial CO₂ partial pressure.

Estimating a shunt value and/or a deadspace value may be performed using the Riley and Cournand 3-compartment model based on the AaP_{O}_{₂} and aAP_{CO}_{₂}.

The present technology also includes a method of detecting pulmonary vascular obstruction (PVO) in a subject suffering from a pulmonary disease, comprising: (a) measuring exhaled oxygen (O₂) and carbon dioxide (CO₂) concentrations over multiple breaths of the subject; (b) determining a mean alveolar O₂ pressure (AP_{O}_{₂}) and a mean alveolar CO₂ pressure (AP_{CO}_{₂}) based on the measurement of step (a); (c) obtaining a single arterial blood sample from the subject; (d) determining an arterial O₂ pressure (aP_{O}_{₂}) and an arterial CO₂ pressure (aP_{CO}_{₂}) from the arterial blood sample of step (c); (e) calculating an alveolar-arterial O₂ pressure difference (AaP_{O}_{₂}) and an arterial-alveolar CO₂ pressure difference (aAP_{CO}_{₂}); (f) estimating a shunt value and/or a deadspace value using the Riley and Cournand 3-compartment model based on the AaP_{O}_{₂} and aAP_{CO}_{₂} of step (e); and (g) detecting PVO based on the shunt value and/or deadspace value of step (f).

The methods disclosed herein may further comprise the step of determining whether the subject has elevated shunt. In some embodiments, a subject has elevated shunt if the subject has a shunt value of at least about 4%, 5%, 6%, 7%, 8%, 9%, or 10%. In some embodiments, a subject has elevated shunt if the subject has a shunt value of at least about 5%.

The methods disclosed herein may further comprise the step of determining whether the subject has elevated deadspace. In some embodiments, a subject has elevated shunt if the subject has a shunt value of at least about 7%, 8%, 9%, 10%, 11%, 12%, 13%, 14%, 15%, 20%, or 25%. In some embodiments, a subject has elevated shunt if the subject has a shunt value of at least about 10%.

Detecting PVO may comprise estimating a deadspace value that is greater than the deadspace of a control subject that does not have PVO. In some embodiments, detecting PVO may comprise estimating a deadspace value that is at least about 5-10%, 10-15%, 15-20%, 20-25%, 25-30%, 30-35%, 35-40%, 40-45%, 45-50%, 50-60%, 60-70%, 70-80%, 80-90%, 90-100%, 100-125%, 125-150%, or 150-200% greater than the deadspace of a control subject. In some embodiments, detecting PVO may comprise estimating a deadspace value that is at least about 5%, 6%, 7%, 8%, 9%, 10%, 15%, 20%, or 25%. In some embodiments, detecting PVO may comprise estimating a deadspace value that is at least about 10%. In some embodiments, detecting PVO may comprise calculating an aAPCO₂ in a subject that is greater than the deadspace in a control subject that does not have PVO. In some embodiments, detecting PVO may comprise calculating an aAPCO₂ in a subject that is at least about 5-10%, 10-15%, 15-20%, 20-25%, 25-30%, 30-35%, 35-40%, 40-45%, 45-50%, 50-60%, 60-70%, 70-80%, 80-90%, 90-100%, 100-125%, 125-150%, or 150-200% greater than the aAPCO₂ in a control subject that does not have PVO.

In some embodiments, the present technology includes detecting PVO in a subject that has pulmonary disease. In some embodiments, detecting PVO in a subject having pulmonary disease comprises estimating a deadspace value that is greater than the deadspace in a control subject that has pulmonary disease but does not have PVO. In some embodiments, detecting PVO in a subject having pulmonary disease comprises estimating a deadspace value that is at least about 5-10%, 10-15%, 15-20%, 20-25%, 25-30%, 30-35%, 35-40%, 40-45%, 45-50%, 50-60%, 60-70%, 70-80%, 80-90%, 90-100%, 100-125%, 125-150%, or 150-200% greater than the deadspace in a control subject that has pulmonary disease but does not have PVO. In some embodiments, detecting PVO in s subject having pulmonary disease comprises estimating a deadspace value that is at least about 5%, 6%, 7%, 8%, 9%, 10%, 15%, 20%, or 25%. In some embodiments, detecting PVO in s subject having pulmonary disease comprises estimating a deadspace value that is at least about 10%. In some embodiments, detecting PVO in a subject having pulmonary disease comprises calculating an aAPCO₂ in a subject that is at least about 5-10%, 10-15%, 15-20%, 20-25%, 25-30%, 30-35%, 35-40%, 40-45%, 45-50%, 50-60%, 60-70%, 70-80%, 80-90%, 90-100%, 100-125%, 125-150%, or 150-200% greater than the aAPCO₂ in a control subject that has pulmonary disease but does not have PVO. In some embodiments, the pulmonary disease is pneumonia. In some embodiments, the pneumonia is caused by COVID-19. In some embodiments, the pulmonary disease is pneumonia.

The present technology includes a method of treating pulmonary infection in a subject in need thereof, comprising: (a) measuring exhaled oxygen (O₂) and carbon dioxide (CO₂) concentrations over multiple breaths of the subject; (b) determining a mean alveolar O₂ pressure (AP_{O}_{₂}) and a mean alveolar CO₂ pressure (AP_{CO}_{₂}) based on the measurement of step (a); (c) obtaining a single arterial blood sample from the subject; (d) determining an arterial O₂ pressure (aP_{O}_{₂}) and an arterial CO₂ pressure (aP_{CO}_{₂}) from the arterial blood sample of step (c); (e) calculating an alveolar-arterial O₂ pressure difference (AaP_{O}_{₂}) and an arterial-alveolar CO₂ pressure difference (aAP_{CO}_{₂}); (f) estimating a shunt value and/or a deadspace value using the Riley and Cournand 3-compartment model based on the AaP_{O}_{₂} and aAP_{CO}_{₂} of step (e); and (g) administering a treatment of the pulmonary infection to the subject based on the shunt value and/or deadspace value of step (f).

In some embodiments, the method further includes determining if the subject has elevated shunt. In some embodiments, if the subject has elevated shunt, a treatment for alveolar flooding, alveolar collapse, or pulmonary edema is administered. A treatment for alveolar flooding or alveolar collapse may be any treatment known by one of skill in the art to be appropriate for treating alveolar flooding, alveolar collapse, or pulmonary edema.

In some embodiments, the method further includes determining if the subject has elevated deadspace. In some embodiments, if the subject has elevated deadspace, the treatment administered may include an anticoagulant. In some embodiments, the anticoagulant is low molecular weight heparin. In some embodiments, if the subject does not have elevated deadspace, the subject is not administered an anticoagulant.

As used herein, “treating” or “treatment” of the pulmonary infection may refer to reducing or eliminating the amount of the infectious organism or particle in the subject, reducing or eliminating the symptoms caused by the pulmonary infection, reducing the length of the pulmonary infection disease course, preventing, delaying, or attenuating the development of a severe reaction to the pulmonary infection, improving the outcome of the subject, or some combination thereof. Treatment may also mean a prophylactic or preventative treatment of a condition. In some embodiments, the pulmonary infection is caused by COVID-19.

As used herein, “reducing” the amount of infectious organism or particle may include decreasing the amount of infectious organism or particle compared to the amount of infectious organism or particle in the subject before the treatment was administered. Reducing the symptoms caused by the infectious organism or particle includes decreasing the amount and/or severity of the symptoms caused by the infectious organism or particle compared to the symptoms before administration of the treatment. As used herein, an “infectious organism or particle” includes bacteria, virus, or fungi. In some embodiments, the infectious organism or particle is COVID-19 virus.

As used herein, “eliminating” the amount of the Infectious organism or particle in the subject includes decreasing the amount of the Infectious organism or particle to an undetectable level. Eliminating the symptoms caused by the Infectious organism or particle includes decreasing the number and severity of symptoms caused by the Infectious organism or particle to a level that is not detectable by the subject. Reducing the length of the Infectious organism or particle disease course includes decreasing the time that it takes to either eliminate the Infectious organism or particle from the subject or eliminate the symptoms caused by the Infectious organism or particle from the subject.

As used herein, “preventing” development of a severe reaction to a pulmonary infection includes blocking the development of one or more symptoms or characteristics of a severe reaction to a pulmonary infection. Symptoms of a severe reaction to a pulmonary infection may include trouble breathing, pain and pressure in the chest, blue lips or face, confusion, an oxygen saturation less than 94%, a ratio of arterial partial pressure of oxygen to fraction of inspired oxygen less than 300 mm Hg, a respiratory rate greater than 30 breaths/min, lung infiltrates greater than 50%, respiratory failure, septic shock, and/or multiple organ dysfunction. Preventing development of a severe reaction to a pulmonary infection also includes preventing the need of additional medical intervention beyond the COVID-19 therapy of the present technology. For example, preventing the development of a severe reaction to a pulmonary infection includes preventing the need for supplemental oxygen, intravenous fluids, diuretics, anticoagulation, increased doses of medications for pre-existing condition(s), and/or antibiotics.

As used herein, “delaying” development of a severe reaction to a pulmonary infection includes preventing the immediate development of one or more symptoms and/or characteristics of a severe reaction to a pulmonary infection compared to a control subject progressor. “Attenuating” the development of a severe reaction to a Pulmonary infection includes slowing the development of one or more symptoms and/or characteristics of a severe reaction to a pulmonary infection compared to a control subject progressor.

As used herein, the word “about” when immediately preceding a numerical value means a range of plus or minus 10% of that value, e.g., “about 50” means 45 to 55, “about 10” means “9 to 11”, etc. Furthermore, the phrases “less than about” a value or “greater than about” or “at least about,” for example, a value should be understood in view of the definition of the term “about” provided herein.

The present technology includes an apparatus for estimating pulmonary shunt and/or deadspace in a subject suffering from a pulmonary disease, comprising: (a) an oxygen (O₂) analyzer configured to analyze oxygen from exhaled gas and arterial blood; (b) a carbon dioxide (CO₂) analyzer configured to analyze carbon dioxide from exhaled gas and arterial blood; (c) a flow meter; and (d) a processor configured to (i) receive an oxygen input from the oxygen analyzer and a carbon dioxide input from the carbon dioxide analyzer, (ii) calculating an alveolar-arterial O₂ pressure difference (AaP_{O}_{₂}) and an arterial-alveolar CO₂ pressure difference (aAP_{CO}_{₂}) from the oxygen input and the carbon dioxide input from step (i); and estimating a shunt value and/or a deadspace value using the Riley and Cournand 3-compartment model based on the AaP_{O}_{₂} and aAP_{CO}_{₂} of step (ii).

### Example 1: Determining Solubility of Gas in Blood and its Relationship to Blood: The Gas Partition Coefficient

In a lung with any degree and pattern of \dot{V}_{A}/\dot{Q} inequality, the properties of any gas undergoing exchange determine to a large extent the magnitude of the interference to its exchange (11, 12). Comparing different gases being exchanged in a given lung, the dominant property of a gas that determines the extent of gas exchange impairment is its solubility, S, in blood (13). The common units of S are mL of gas per 100 mL of blood per mmHg partial pressure of the gas. Solubility can also be expressed as the blood to gas partition coefficient, commonly denoted by the Greek letter A. A for any gas is the ratio of its concentration in blood to its concentration in gas with which it is in equilibrium. Because A is a ratio of two concentrations, it is a dimensionless number. When S is expressed in mL/100 mL/mmHg, the numerical relationship between S and A is as follows:

$$λ = S \times ( PB - {P H}_{2} O ) / 100 \qquad ( 1 )$$

Here PB is barometric pressure and PH₂O is saturated water vapor pressure, both in mmHg. Multiplying S by (PB−PH₂O) converts its units to mL/100 mL/atmosphere, and then dividing S by 100 further converts its units to mL/mL/atmosphere, which defines λ as the mL of gas in 1 mL of blood when, in the gas phase, its concentration is 1 mL/mL. Under standard conditions, i.e., PB=760 mmHg and at body temperature of 37° C. where PH₂O=47 mmHg, the outcome is:

$$λ = 7.13 \times S \qquad ( 2 )$$

For inert gases (gases that are chemically nonreactive with blood and tissue components) and which are therefore carried in blood only in physical solution (i.e., dissolved), either S or λ is sufficient to characterize the relationship between partial pressure and concentration over any range of values.

However, for O₂ and CO₂, the relationships between partial pressure and concentration in blood are known to be nonlinear, and a single value of S or λ is thus insufficient to describe their partial pressure-concentration relationships quantitatively with accuracy. However, for the analysis to follow, it will be very useful in conceptual development to begin by defining the O₂ (or CO₂) dissociation curves by their average slopes between arterial and mixed venous blood, thereby assigning them average values of S and A.

Focusing first on O₂, where breathing ambient air, normal arterial blood has a PO₂ of 100 mmHg and an O₂ concentration of 20 mL/100 mL blood and where normal mixed venous blood has a PO₂ of 40 mmHg and an O₂ concentration of 15 mL/100 mL blood, it can be seen that the average slope of the O₂ dissociation curve in the normal range becomes (20-15)/(100-40) mL O₂/100 mL blood/mmHg. This comes to 5/60 or 0.083 mL/100 mL/mmHg. In the present context, this number becomes the value of S for O₂, and multiplying by (PB−PH₂O)/100 (i.e., by 7.13 under standard conditions) yields an effective blood:gas partition coefficient for O₂ of ˜0.6.

Turning to CO₂, normal arterial blood has a PCO₂ of 40 mmHg and a CO₂ concentration of 48 mL/100 mL blood and normal mixed venous blood has a PCO₂ of 45 mmHg and a CO₂ concentration of 52 mL/100 mL blood. From these numbers, it can be seen that the average slope of the CO₂ dissociation curve in the normal range becomes (52-48)/(45-40) mL CO₂/100 mL blood/mmHg. This comes to 4/5 or 0.80 mL/100 mL/mmHg. In the present context, this number becomes the value of S for CO₂, and multiplying by 7.13 yields an effective blood:gas partition coefficient for CO₂ of ˜6.

The average slope of the CO₂ dissociation curve is ˜10 times the average slope of the O₂ dissociation curve, and the analysis continues considering O₂ to behave like a gas with λ=0.6 and CO₂ to behave like a gas with λ=6. The utility of these simplifying approximations will become apparent in the analysis that now follows.

### Example 2: Measuring Gas Exchange in the Presence of \dot{V}A/\dot{Q}Inequality: The Partition Coefficient

Ventilation/perfusion (\dot{V}_{A}/\dot{Q}) inequality is defined as variation in the local \dot{V}_{A}/\dot{Q} ratio across the lungs. In patients with lung disease, it is common knowledge that \dot{V}_{A}/\dot{Q}inequality is a dominant consequence of structural and functional pulmonary abnormalities, causing interference to gas exchange manifest as arterial hypoxemia, sometimes also with hypercapnia. Research using both imaging tools and the multiple inert gas elimination technique has revealed that \dot{V}_{A}/\dot{Q} inequality is often manifest as a distribution of lung units over a wide range of \dot{V}_{A}/\dot{Q}ratios. However, for conceptual development of the present technology, there is major advantage to simplifying the lungs to a two-compartment structure where one compartment, or unit, has a \dot{V}_{A}/\dot{Q}ratio in the normal range and the other is abnormal with a \dot{V}_{A}/\dot{Q}ratio that is either lower or higher than that of the normal unit.

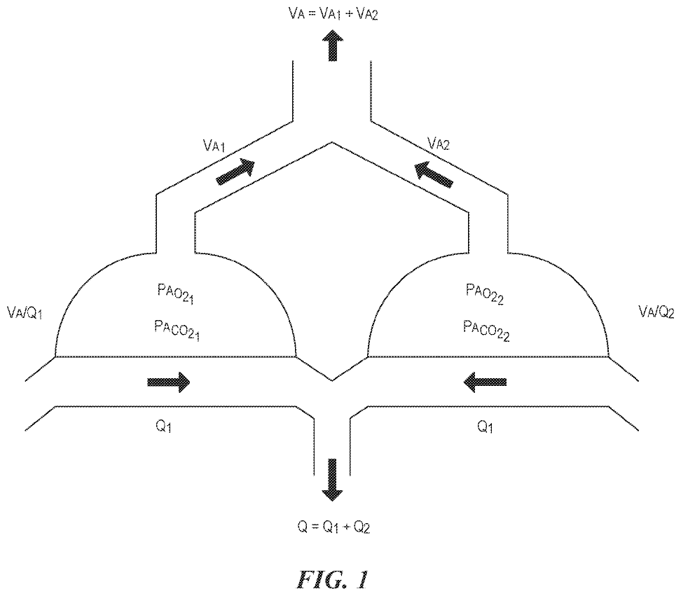

Suppose the lungs are now imagined as the simplest model of \dot{V}_{A}/\dot{Q} inequality using a two-compartment model (FIG. 1). Here one side (FIG. 1, left) has alveolar ventilation designated \dot{V}A₁ and blood flow designated \dot{Q}₁, whereas the other side (FIG. 1, right) has values denoted \dot{V}A₂ and \dot{Q}₂. Total alveolar ventilation is \dot{V}λ=\dot{V}A₁+\dot{V}A₂, whereas total blood flow \dot{Q}=\dot{Q}₁+\dot{Q}₂. Gas exchange is presumed to occur in a steady state.

Suppose further that gases are being eliminated from the blood (i.e., as for CO₂) and exhaled to the atmosphere (rather than being moved from alveolar gas into blood). Mathematically, this makes the algebra that follows simpler, because inspired levels of the gas can be set to zero and thus ignored. The direction of exchange, however, is immaterial to the result. Using the principle of mass conservation in pulmonary gas exchange, the following relationship can be derived by relating ventilation and blood flow in an alveolus to the partial pressures of the gas in alveolar gas (PA), end-capillary blood (Pec), and mixed venous blood (Pv) and to the partition coefficient (λ) of the gas (14, 15):

$${PA}_{1} = {Pec}_{1} = Pv \times λ / ( λ + \frac{\dot{V} {A}_{1}}{{\dot{Q}}_{1}} ) for unit 1 \qquad ( 3 )$$

$$and$$

$${PA}_{2} = {Pec}_{2} = Pv \times λ / ( λ + \frac{\dot{V} {A}_{2}}{{\dot{Q}}_{2}} ) for unit 2 \qquad ( 4 )$$

Here alveolar and end-capillary gas tensions are taken to be identical in each unit, although clearly different between units because \dot{V}A₁/\dot{Q}₁ does not equal \dot{V}A₂/\dot{Q}₂. Alveolar end capillary equivalence implies that alveolar-capillary diffusion equilibrium does occur within the transit of each red blood cell through the microcirculation of the lungs.

Now defining R₁ as Pec1/Pv (the retention of unit 1) and E₁ as PA₁/Pv (the excretion of unit 1), and similarly applying this simplification to unit 2, the two equations become:

$${R}_{1} = {E}_{1} = λ / ( λ + \frac{\dot{V} {A}_{1}}{{\dot{Q}}_{1}} ) for unit 1 \qquad ( 5 )$$

$$and$$

$${R}_{2} = {E}_{2} = λ / ( λ + \frac{\dot{V} {A}_{2}}{{\dot{Q}}_{2}} ) for unit 2 \qquad ( 6 )$$

Equation 5 states that in gas exchange unit 1, the fraction R₁ of the gas delivered to that unit in the pulmonary arterial blood which remains in the end-capillary blood of that unit after undergoing pulmonary gas exchange is the ratio of the partition coefficient of the gas to the sum of the partition coefficient and the \dot{V}_{A}/\dot{Q}ratio of the unit. Equation 6 makes the identical statement for the second unit. The equations show that R₁ and R₂ will be lower for a poorly soluble gas (with low λ) than for a highly soluble gas (with high λ) in any gas exchange unit, and higher for any gas when the unit's \dot{V}_{A}/\dot{Q}ratio is low compared with when its \dot{V}_{A}/\dot{Q}ratio is high.

Referring to FIG. 1, suppose the two \dot{V}_{A}/\dot{Q}compartments are connected in parallel by both airways and blood vessels as shown. Their exhaled airstreams will mix and their end capillary blood streams will also mix as ventilation and perfusion continue. Because of this, the mixed exhaled gas will have a ventilation-weighted average partial pressure, normalized to Pv, (and termed PE) given by:

$$PE = ( {E}_{1} \times \dot{V} {A}_{1} + {E}_{2} \times \dot{V} {A}_{2} ) / ( \dot{V} {A}_{1} + \dot{V} {A}_{2} ) \qquad ( 7 )$$

Symmetrically, because the two bloodstreams also mix to form the systemic arterial blood after leaving the alveolar region, the mixed arterial blood will have a perfusion weighted average partial pressure (Pa), also normalized to Pv, given by:

$$Pa = ( {R}_{1} \times {\dot{Q}}_{1} + {R}_{2} \times {\dot{Q}}_{2} ) / ( {\dot{Q}}_{1} + {\dot{Q}}_{2} ) \qquad ( 8 )$$

Just as for the alveolar-arterial difference for O₂, the difference between Pa and PE expresses the degree of interference to gas exchange (of a gas with partition coefficient λ in this particular two-unit lung with ventilation and blood flow distributed as described). Hence:

$$Pa - PE = ( {R}_{1} \times {\dot{Q}}_{1} + {R}_{2} \times {\dot{Q}}_{2} ) / ( {\dot{Q}}_{1} + {\dot{Q}}_{2} ) - ( {E}_{1} \times \dot{V} {A}_{1} + {E}_{2} \times \dot{V} {A}_{2} ) / ( \dot{V} {A}_{1} + \dot{V} {A}_{2} ) \qquad ( 9 )$$

The terms on the right side of Equation 9 contain R₁ and E₁, both of which equal

$$λ / ( λ + \frac{\dot{V} {A}_{1}}{{\dot{Q}}_{1}} )$$

and also R₂ and E₂, both of which equal

$$λ / ( λ + \frac{\dot{V} {A}_{2}}{{\dot{Q}}_{2}} )$$

In sum, the unique terms in Equation 9 are only five in number: \dot{V}A₁, \dot{V}A₂, \dot{Q}₁, \dot{Q}₂, and λ. Therefore, for a given set of values of \dot{V}A₁, \dot{V}A₂, \dot{Q}₁ and \dot{Q}₂, the arterial-alveolar gas partial pressure difference, Pa−PE, is a unique function of λ.

In fact, when \dot{V}A₁, \dot{V}A₂, \dot{Q}₁, and \dot{Q}₂ are nonzero themselves, the equations show that if A were zero, the arterial-alveolar gas partial pressure difference must be zero as both R₁ and R₂ fall to zero. Similarly, when A is infinitely high, the arterial-alveolar gas partial pressure difference must also be zero because R₁=R₂=1.0. In between these extremes, differential calculus can be used to show that there is a unique value of A for which the arterial-alveolar gas partial pressure difference will be at a maximum. This requires differentiating Equation 9 with respect to λ and solving for the value of A that makes the differentiated equation zero. That process defines the value of λ at which Pa−PE is maximal (Δₘₐₓ).

This unique value of λₘₐₓ must be a function of the values of \dot{V}A₁, \dot{V}A₂, \dot{Q}₁, and \dot{Q}₂ because they are the only other terms than λ in Equation 9. When this calculus is performed, the outcome is that the arterial-alveolar gas partial pressure difference will be at a maximum when:

$$λ max = \sqrt{( \frac{\dot{V} {A}_{1}}{{\dot{Q}}_{1}} ) \times ( \frac{\dot{V} {A}_{2}}{{\dot{Q}}_{2}} )} \qquad ( 10 )$$

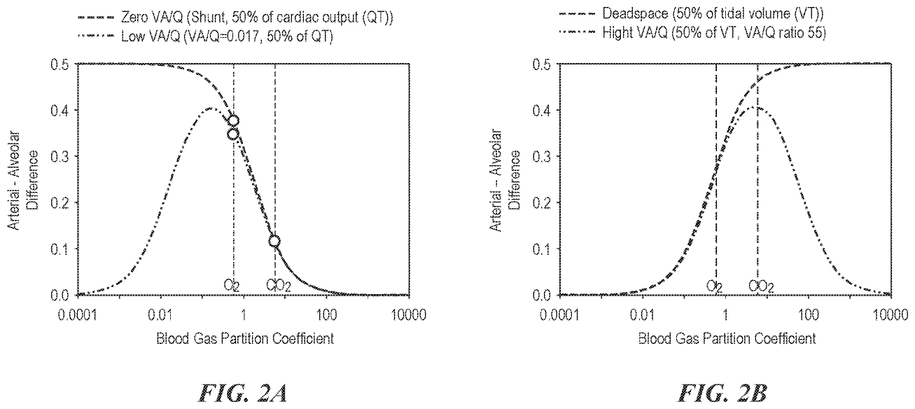

FIG. 2 shows the arterial-alveolar gas partial pressure difference calculated using Equation 9 over a wide range of A for four model lungs, two in each panel. FIG. 2A describes alveolar-arterial difference outcomes using Equation 9 for a wide range of partition coefficients for two models having (1) 50% shunt (\dot{V}_{A}/\dot{Q}=0) perfusion and 50% perfusion of normal lung (\dot{V}_{A}/\dot{Q}=1; upper line) and (2) 50% low \dot{V}_{A}/\dot{Q}(0.017) perfusion and 50% normal perfusion (\dot{V}_{A}/\dot{Q}=1; lower line).

The values of the average slopes of the O₂ and CO₂ dissociation curves expressed as partition coefficients as discussed above are indicated by the vertical dashed lines. Although the lower and upper curves can be seen to differ markedly at very low partition coefficients, at the partition coefficients representing O₂ and CO₂, there is little difference between them: whether the lung has low \dot{V}_{A}/\dot{Q} areas or shunt makes little difference to O₂ or CO₂ exchange (when ambient air is breathed, as proposed in this technology).

FIG. 2B describes alveolar-arterial difference outcomes using Equation 9 for a wide range of partition coefficients for two models having (1) 50% deadspace (\dot{V}_{A}/C=infinitely high) ventilation and 50% ventilation of remaining normal lung (\dot{V}_{A}/C=1; upper line) and (2) 50% high \dot{V}_{A}/\dot{Q} (55) ventilation and 50% ventilation of remaining normal lung (\dot{V}_{A}/\dot{Q}=1; lower line).

The values of the average slopes of the O₂ and CO₂ dissociation curves are again expressed as partition coefficients and are indicated by the vertical dashed lines. Although the lower and upper curves can be seen to differ markedly at very high partition coefficients, for O₂ and CO₂ there is, as with FIG. 2A, little difference between them: whether the lung has high \dot{V}_{A}/\dot{Q} areas or actual unperfused deadspace makes little difference to O₂ or CO₂ exchange.

Thus a lung with low—or zero—\dot{V}_{A}/\dot{Q} ratios affects a gas with partition coefficient equivalent to that of O₂ more than one with partition coefficient equivalent to that of CO₂, and that a lung with high—or infinite—\dot{V}_{A}/\dot{Q} areas affects CO₂ more than O₂. Either a shunt or a lesion with low \dot{V}_{A}/\dot{Q} areas will affect both O₂ and CO₂, whereas high \dot{V}_{A}/C regions and deadspace also affect both gases. This means that O₂ cannot be used alone to define shunt (or low \dot{V}_{A}/\dot{Q} areas) and CO₂ cannot be used alone to define deadspace (or high \dot{V}_{A}/\dot{Q} areas). The power of this analysis lies in the simultaneous use of the two gases, as will be further developed below.

The ensuing examples exploit these principles to apportion shunt and deadspace in the lungs of any individual subject by measuring the alveolar-arterial partial pressure differences for both O₂ and CO₂ and calculating the size of the shunt and deadspace necessary to explain those differences. The framework for this approach is the three compartment analysis developed by Riley and Cournand (16) whereby the lung is divided into three virtual compartments: one contains all of the shunt, the second contains all of the deadspace, and the third contains all of the nonshunt blood flow and nondeadspace ventilation. In this model, perfusion of low (but nonzero) \dot{V}_{A}/\dot{Q}regions is expressed as equivalent shunts. Correspondingly, ventilation of high (but not infinitely high) \dot{V}_{A}/\dot{Q}regions is expressed as equivalent deadspace. Even though both shunt and low \dot{V}_{A}/\dot{Q}areas may be present, the analysis provides a value for the size of the shunt required to fully explain arterial PO₂ and PCO₂, and the size of the deadspace required to fully explain the expired P_{O}_{₂} and PCO₂. To accomplish this, the quantitative relationships between (1) deadspace and shunt and (2) arterial-alveolar partial pressure differences for O₂ and CO₂ must first be established.

### Example 3: The Alveolar-Arterial P_{O}_{₂} Difference and the Arterial-Alveolar PCO₂ Differences in Lungs with Shunt and Deadspace

Although the preceding analysis provides an analytical explanation of why the exchange of any gas becomes differentially affected depending on the type of \dot{V}_{A}/\dot{Q}inequality present in the lungs and the partition coefficient of the gas, the approximation of the carriage of both O₂ and CO₂ in blood by a single hypothetical average “partition coefficient” must be addressed to better reflect the fact that both O₂ and CO₂ possess nonlinear dissociation curves. This means using their actual dissociation curves, rather than their average dissociation curve slopes. Fortunately, this can readily be accomplished, as follows.

In 1969, West (17) described a set of algorithms that would calculate the mixed (systemic) arterial and mixed exhaled alveolar P_{O}_{₂} and PCO₂ values in a lung of any selected degree of \dot{V}_{A}/\dot{Q}inequality. The actual O₂ and CO₂ dissociation curves described by Kelman (18-20) were employed in this computer program, which allows for their chemical interaction. In this way, the linear approximation to their dissociation curves used in the preceding theoretical section, while conceptually essential, was avoided in the application. Factors affecting the shape and position of both dissociation curves (in particular, hemoglobin concentration and P50, base excess and temperature) are accounted for quantitatively.

Moreover, although West depicted inequality by means of a log-normal multicompartmental \dot{V}_{A}/\dot{Q}distribution model, his algorithm is easily pared down to the simpler Riley and Cournand 3-compartment model of shunt, deadspace, and a normal compartment, which was used in the present study. From a single measurement of alveolar and arterial PO₂ and PCO₂ with the patient breathing ambient air, the information available is insufficient to describe the shape and position of the entire \dot{V}_{A}/\dot{Q}distribution but is sufficient to determine shunt and deadspace in the Riley construct. Karbing et al. recognized this and used multiple measurements over a range of inspired O₂ concentrations to discriminate better areas of low \dot{V}_{A}/\dot{Q}ratio from shunt areas of zero \dot{V}_{A}/\dot{Q}ratio (21-23).

Inputs to West's algorithm are the values of ventilation and blood flow in each compartment (and hence their sums, i.e., total alveolar ventilation and pulmonary blood flow). Ancillary input data are O₂ consumption, CO₂ production, inspired O₂ and CO₂ concentrations, [Hb], Hb P50, barometric pressure, body temperature, and base excess. Outputs from West's algorithm are the alveolar-arterial differences for O₂ and CO₂, and the corresponding values of shunt and deadspace that in combination account for both alveolar-arterial PO₂ and PCO₂ differences.

To show the relationships between arterial-alveolar differences for O₂ and CO₂ and the corresponding values of shunt and deadspace, three 3-compartmental models of \dot{V}_{A}/\dot{Q} inequality were constructed, and West's algorithm was utilized. One—modeling only the expected development of regions of deadspace from diffuse microvascular obstruction-employed a lung with a compartment of normal \dot{V}_{A}/\dot{Q} (˜1) plus a second compartment of infinitely high \dot{V}_{A}/\dot{Q}ratio, with its ventilation ranging from 0% to 50% of tidal volume. Here, for the third compartment (shunt), perfusion was set to zero to determine how deadspace alone affected the arterial-alveolar difference for both gases. A second model performed the mirror-image calculations using a normal compartment and one with \dot{V}_{A}/\dot{Q}ratio=0. Here, the third compartment (deadspace) was assigned zero ventilation so as to model the pure effects of a shunt on alveolar-arterial differences. The third model combined various amounts of shunt plus deadspace and determined the alveolar-arterial differences over these ranges.

The alveolar-arterial P_{O}_{₂} difference depicted in the above is not the classical difference commonly derived from the alveolar gas equation. Such an approach would not be valid in the present context, because the alveolar gas equation specifically assumes that the alveolar PCO₂ can be approximated by the arterial PCO₂, which is clearly untrue whenever there is \dot{V}_{A}/\dot{Q}inequality (see FIG. 2). The appropriate alveolar values to compare to arterial are the mixed alveolar values of both O₂ and CO₂, which are the ventilation-weighted averages of the compartmental values.

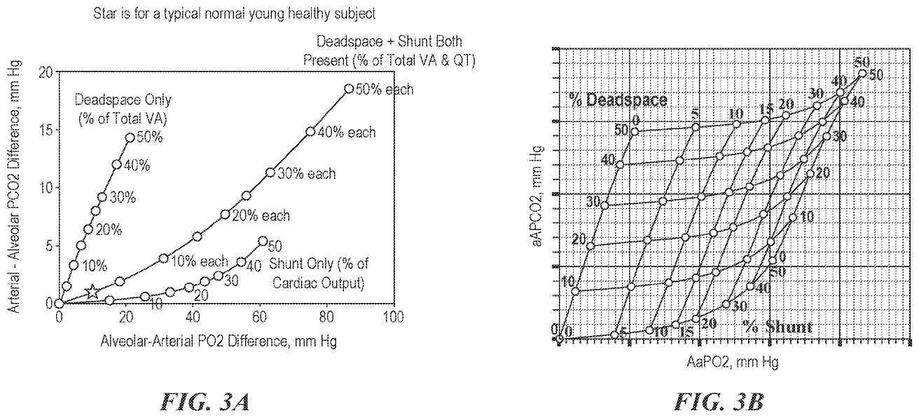

FIG. 3A plots the arterial-mixed alveolar PCO₂ differences provided by West's algorithm against those for O₂ for the three models (shunt only; deadspace only; both shunt and deadspace). Results for values of shunt and deadspace from 0% to 50% are shown. This figure provides the fundamental framework of the proposed approach to determining the existence of areas of high \dot{V}_{A}/\dot{Q}ratio in patients with COVID. Where a patient's measured PO₂ and PCO₂ alveolar-arterial differences lie on this framework figure enables quantification of the amounts of shunt and deadspace, singly or in combination. If the sole gas exchange defect is vascular obstruction causing deadspace, the data should lie somewhere along the “deadspace only” line, depending on the extent of the obstruction. If the sole defect is the existence of shunt, the patient data should lie along the lower line; when both types of lesions coexist, the data would lie in between these two lines. The “deadspace and shunt” line shows the example of what would be seen if there were equal amounts of shunt and deadspace present. Importantly, in each of the three models, both gases are affected, and the relative amount by which each gas is affected provides insight to the amount of shunt and deadspace. This diagram therefore allows for estimates of both shunt and deadspace simultaneously. It recognizes that shunt affects CO₂ and must be allowed for. Correspondingly it recognizes that deadspace affects O₂ which must also be allowed for.

FIG. 3B extends the calculations of FIG. 3A to form a complete grid depicting how any combination of shunt and deadspace affects the alveolar-arterial partial pressure differences for O₂ and CO₂. This grid may then be used in reverse to identify the amounts of shunt and deadspace corresponding to any measured pair of values of alveolararterial differences for O₂ and CO₂.

To apply these concepts to individual patients as accurately as possible, the above-mentioned ancillary variables (O₂ consumption, CO₂ production, inspired O₂ and CO₂ concentrations, [Hb], Hb P50, barometric pressure, body temperature and base excess) are used in executing West's algorithm, along with total alveolar ventilation, pulmonary blood flow (assumed equal to cardiac output). This means that the grid of FIG. 3B is in effect calculated uniquely for each patient using measured values of the ancillary variables. All of these variables are available from the proposed measurements, either from the expired gas or the arterial blood samples, with the exception of Hb P50 and cardiac output. In the majority of patients without hemoglobinopathy, Hb P50 lies within a narrow range around 27 mmHg (24-26), and the sensitivity of the estimated shunt and deadspace to variation in P50 given the measured alveolar-arterial differences was explored. Similarly, absent its direct measurement, cardiac output was estimated using a round number formula (cardiac output=5×O₂ consumption+5, with both O₂, consumption and cardiac output in 1/min) that has been shown to closely describe how cardiac output varies linearly with O₂ consumption (27, 28) so that sensitivity to this variable could also be explored.

### Example 4: Determination of Mixed Alveolar PO₂ and PCO₂ Values from Exhaled Breath Profiles

As mentioned, the alveolar-arterial PO₂ and PCO₂ differences used in the present approach are not those derived from the standard alveolar gas equation, because the difference for CO₂ is explicitly assumed to be zero when that equation is used. Rather, the arterial values for each gas are compared with their mixed alveolar values. This raises the question of how to determine mixed alveolar values, averaged over the whole breath.

In 1952, Arthur DuBois and his coworkers addressed the issue of how alveolar PO₂ and PCO₂ varied throughout the respiratory cycle (29-32). They showed that alveolar PCO₂ fell and PO₂ rose during inspiration, and then reversed direction during expiration, as would be expected from tidal inspiration of air high in O₂ and low in CO₂. This led to the notion of an oscillating alveolar (and blood) PO₂ and PCO₂ about their means during steady state breathing. When sampling arterial blood, the aspiration procedure may be intentionally prolonged over at least 2-4 breaths so as to obtain a de facto mean arterial blood level for each gas. This in turn requires that if alveolar gas is to be compared with arterial blood in terms of both PO₂ and PCO₂, the best estimates of alveolar PO₂ and PCO₂ for this comparison would be their mean values averaged over the entire respiratory cycle.

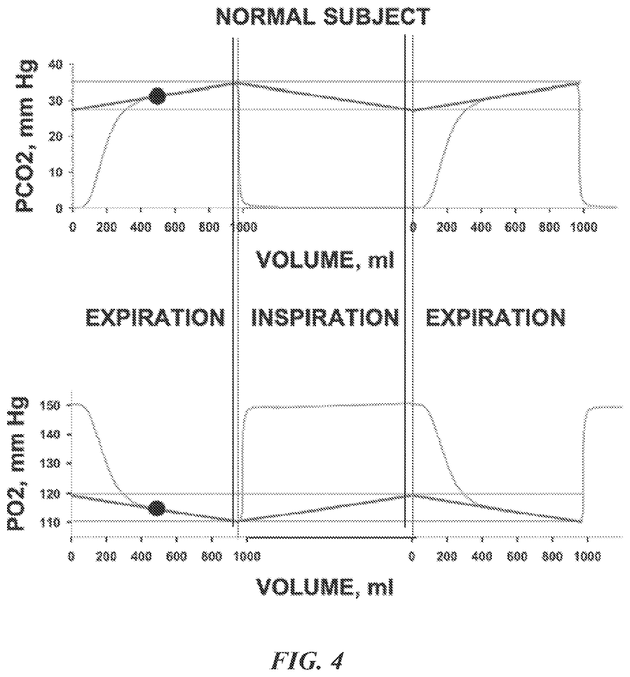

There is an immediate practical problem with this logical conclusion: One can only observe alveolar gas during expiration, and even then, only after the anatomic deadspace gas has been washed out. During inspiration, sampling at the mouth records only inspired air. It would require a probe in the alveoli to record alveolar P_{O}_{₂} and P_{CO}_{₂} over the whole cycle. However, the observable portion of the alveolar gas (after anatomic deadspace washout during expiration) is well known to follow a straight, almost flat, trajectory in health (33, 34). FIG. 4 portrays (curved tracings) two exhalations with an inhalation in between for both O₂ and CO₂ from a normal subject to illustrate the issues. The first ˜100 mL of expirate is low in CO₂ and high in O₂, reflecting “anatomic deadspace” gas exhaled first from the conducting airways (i.e., gas at or near inspired levels), transitioning over the next ˜200 mL to the alveolar plateau, which is in this case a linear, slightly sloped line (up for CO₂, down for O₂) continuing until the end of the expiration. The slope in healthy subjects, such as that in FIG. 4, is due mostly to continuing gas exchange as CO₂ keeps moving from blood to gas as expiration continues, causing alveolar PCO₂ to rise. The opposite holds for O₂ being moved from alveolar gas into capillary blood, causing PO₂ to fall.

The alveolar plateau is essentially linear over the entire post-deadspace washout portion. This allows the reasonable inference that in a steady state with constant tidal volume, breathing frequency, metabolic rate, and cardiac output, linearity in actual alveolar PO₂ and PCO₂ change would continue throughout the respiratory cycle, even though it cannot be seen at the mouth. This notion is illustrated in FIG. 4 where both the visible and invisible but presumed alveolar values are indicated by the straight black lines drawn as a tangent to the alveolar plateau for each gas. As long as the alveolar gas levels change linearly, the mean alveolar values are, by simple geometry, those values at the volume midpoint of the exhalation (solid black circles in FIG. 4). It is therefore proposed to use the alveolar P_{O}_{₂} and P_{CO}_{₂} at the midpoint of exhalation as the best estimate of the mixed alveolar values of P_{O}_{₂} and P_{CO}_{₂} averaged over the respiratory cycle. It is these values that would then be compared with the measured arterial P_{O}_{₂} and P_{CO}_{₂} values, so as to derive the alveolar-arterial differences to plot on the framework of FIG. 3B. Note that end-tidal P_{O}_{₂} and P_{CO}_{₂} values would be inappropriate for such a comparison because they provide the highest rather than the mean P_{CO}_{₂} and lowest rather than the mean P_{O}_{₂} throughout the respiratory cycle.

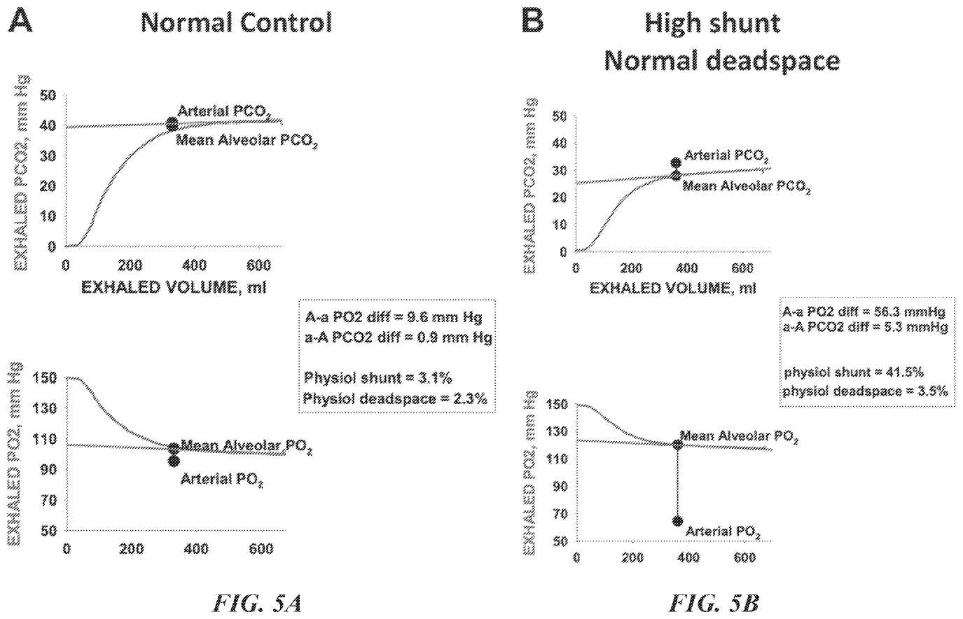

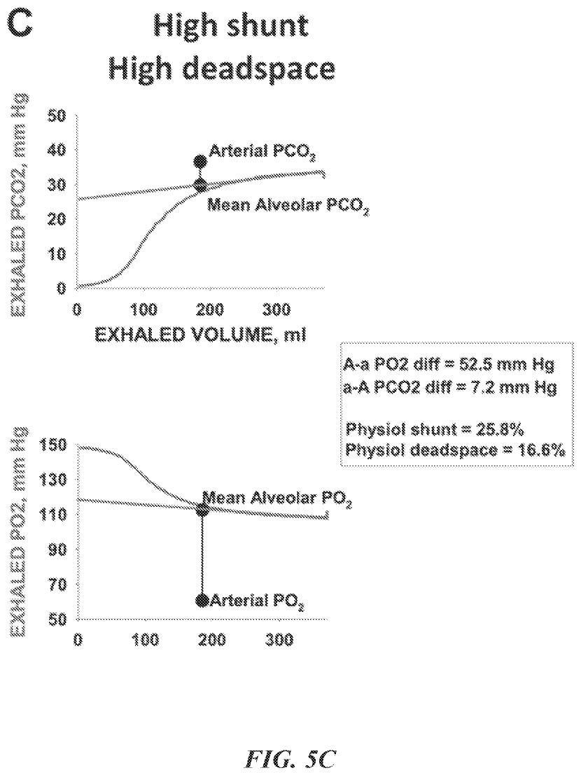

FIG. 5 shows the application of this methodology for one normal subject (FIG. 5A) and two patients with COVID-19 (FIGS. 5B and 5C). These single, expired gas, tracings indicate proof of concept, showing an essentially linear alveolar plateau for both gases in each case, enabling estimation of the mean alveolar PO₂ and PCO₂.

FIG. 5 raises the questions of sensitivity and specificity of the proposed approach. Sensitivity requires knowing the 95% upper confidence limit of normal for both physiological shunt and deadspace. Based on data from the multiple inert gas elimination technique used in subjects breathing ambient air (35), the 95% upper confidence limits for physiological shunt and physiological deadspace are 5% and 10%, respectively. The upper limit for shunt is less that of deadspace because of the flatness of the O₂-Hb dissociation curve in the normal range and because there is slightly more dispersion of ventilation than blood flow. Values above these limits are therefore highly unlikely to be false-positive results, whereas values below those limits are considered to be within the normal range. On the other hand, specificity requires an independent method for assessing the presence of pulmonary vascular obstruction causing high \dot{V}A/\dot{Q}regions, and in the current context, there are no adequate approaches when the vascular lesions are likely many and small. This makes specificity unable to be addressed currently. That said, it is well established that areas of high \dot{V}A/\dot{Q}ratio interfere especially with CO₂ exchange, and so the inference of such regions from elevated arterial-alveolar PCO₂ values should not be in question.

As mentioned, West's algorithm was executed using each patient's own measured ancillary variables. Both Hb P50 and cardiac output, however, were not measured and were thus estimated. Hb P50 was assumed to be 26.8 mmHg, and cardiac output as 5×O₂ consumption+5 (27, 28) with both O₂ consumption and cardiac output in l/min. Because these variables had to be estimated, sensitivity of derived shunt and deadspace to variation in Hb P50 and to variation in cardiac output was determined.

Sensitivity analysis based on the three example cases shown in FIG. 5 was performed with varying cardiac output by ±25% from its assumed value and varying Hb P50 by ±2 mmHg. There was an insignificant sensitivity of deadspace to either condition. Shunt showed a small degree of sensitivity to Hb P50 that increased as shunt fraction increased. For example, the case in FIG. 5C showed a variation in shunt of three percentage points at the extremes of the Hb P50 range around a value of 26% shunt. Shunt estimates were affected more by uncertainty in cardiac output, but when shunt was modest, the uncertainty was small. Although there is evident sensitivity to cardiac output when shunt is large (e.g., FIG. 5C), even this was modest. In this case, there was a variation of approximately ±4 percentage points for a 25% change in cardiac output (i.e., when cardiac output was almost doubled from 6 to 10 L/min).

The present technology provides a theoretical foundation and practical method for the use of simultaneous measurements of P_{O}_{₂} and P_{CO}_{₂} in alveolar gas and arterial blood to estimate both the percentage shunt and the percentage deadspace. The new analyses point out how both shunt and deadspace affect both O₂ and CO₂ in their exchange, so that using O₂ measurements alone to define shunt and CO₂ measurements alone to define deadspace (as is common practice) is insufficient. Rather, the present analysis points out that O₂ and CO₂ measurements together are needed to simultaneously estimate shunt and deadspace. Additionally, the present technology underscores that equating arterial and alveolar PCO₂, as is the norm when using the conventional alveolar gas equation, undermines the proposed approach because with either shunt or deadspace, the arterial-alveolar PCO₂ difference is substantial.

The present technology answers the question of whether, in patients with lung disease, especially COVID-19, such patients' hypoxemia and arterial PCO₂ can be explained fully by shunt, or whether there are additional gas exchange defects. In particular, the present technology can determine whether the percent of deadspace is in excess of that expected from shunt alone. Deadspace in excess of that expected from shunt alone in spontaneously breathing patients suggests the presence of high \dot{V}A/\dot{Q}areas indicative of pulmonary vascular obstruction. Imaging and catheter-based approaches to determining small vessel obstruction are problematic in the context of clinical COVID-19.

The use of the respiratory gases O₂ and CO₂ rather than the multiple inert gas elimination technique (13) both answers the question about percent of deadspace and is feasible at the bedside, even in the patient with COVID-19. Measurement of arterial blood gas levels is clinically commonplace; measurement of exhaled gas concentrations requires the patient to provide no more than 10 breaths (over ˜30 s) under steady-state conditions, with collection of a single arterial blood sample during this time. During this period, continuous measurement of O₂ and CO₂ at the mouth provides for subsequent calculation of O₂ and CO₂ levels at mid-expiration (see FIG. 4) along with total alveolar ventilation, O2 consumption, and CO₂ production. Measurement of inhaled and exhaled O₂ and CO₂ at the mouth at high frequency (100 Hz) is done routinely in cardiopulmonary exercise testing (CPET). With the raw data, mean alveolar O₂ and CO₂ levels then are determined as the values of [O₂] and [CO₂] at the point halfway through exhalation.

Since the derivation of shunt and deadspace from alveolar and arterial P_{O}_{₂} and P_{CO}_{₂} is based on steady-state relationships among these variables, it is preferred to ensure a steady state during data collection. A metronome may be used to encourage constant respiratory rate and monitor constancy (±1 mmHg) of end-tidal P_{O}_{₂} as an indicator of an adequate steady state. Tidal volume should be sufficient in size to produce an alveolar plateau adequate in length for inscribing a least-squares best-fit line to its linear portion. The methods of the present technology may be used at a higher fraction of inspired oxygen (FI_{O}_{₂}). As FI_{O}_{₂} is raised, areas of low \dot{V}_{A}/\dot{Q}ratio that have an alveolar P_{O}_{₂} close to mixed venous when FI_{O}_{₂} is 0.21 will develop a progressive increase in alveolar P_{O}_{₂} as FI_{O}_{₂} is raised. This will increase experimental complexity because FI_{O}_{₂} needs to be known to employ the methodology. The patients may be spontaneously breathing or mechanically ventilated. If the present technology used in a ventilated patient shows a high alveolar deadspace, it may represent either pulmonary vascular obstruction or a functional outcome of the ventilatory strategy.

In some embodiments, the relationship between alveolar-arterial differences and shunt/deadspace may be modulated by factors such as body temperature, [Hb], and acid-base status. These factors are routinely available from the blood gas sample (noting that body temperature is required for proper interpretation of arterial P_{O}_{₂} and P_{CO}_{₂}) and are readily incorporated into the calculation of shunt and deadspace (via the algorithm of West). Although cardiac output and Hb P50 are both not commonly measured (but can each affect the calculated shunt for a given pair of alveolar-arterial differences), they can be determined experimentally and relatively noninvasively from standard techniques. But even without their measurement, uncertainty in shunt is generally small and there is substantially no effect on deadspace.

In summary, the present technology includes a bedside approach applicable in health and pulmonary diseases, such as COVID-19 and other acute (or chronic) respiratory illnesses, that simultaneously samples exhaled gas and arterial blood over several breaths to determine the alveolar-arterial differences for both PO₂ and PCO₂. The methodology employs commonly available instrumentation for expired gas and arterial blood analysis. The PO₂ and PCO₂ differences are then used to determine shunt and deadspace, which reflect the extent of poorly ventilated (or unventilated) regions subject to alveolar filling and poorly perfused (or unperfused) regions subject to vascular obstruction.

### Example 5: Intrapulmonary Shunt and Alveolar Deadspace in a Cohort of Patients with COVID-19 Pneumonitis and Early Recovery

Since the beginning of 2020, over half a billion people have been diagnosed with COVID-19, a disease caused by infection with the novel severe acute respiratory syndrome coronavirus 2 (SARS-CoV-2). Many have required hospitalisation, with 10-20% of those requiring intensive care, mainly due to respiratory compromise (36). Worldwide there have been more than 6 million deaths.

Patients with early COVID-19 respiratory failure present with hypoxaemia and hyperventilation (37-41). The hypoxaemia is likely related to ventilation/perfusion (\dot{V}A/\dot{Q}) mismatch and in particular to increased intrapulmonary shunt arising from alveolar filling with fluid or cellular debris. However, pathology reports from COVID-19 infected lungs also frequently demonstrate pulmonary vasculature involvement, including severe endothelial injury, widespread thrombosis with microangiopathy and new vessel growth (42-44). Pulmonary vessel microembolism reduces capillary blood flow, thus generating areas of high \dot{V}A/\dot{Q} and promoting increased alveolar deadspace.

In early COVID-19 pneumonitis, hypoxaemia may be associated with both increased intrapulmonary shunt and increased alveolar deadspace. Using bedside measurement of exhaled partial pressure of oxygen (PO₂) and carbon dioxide (PCO₂) combined with arterial blood gas measurements, alveolar-arterial partial pressure differences were measured for both oxygen (PA-aO₂) and carbon dioxide (Pa-ACO₂), from which both intrapulmonary shunt and alveolar deadspace values were then determined using a novel computational model (45).

Subjects 30 patients admitted to Danderyd Hospital in Stockholm, Sweden who were >18 years old and PCR-positive for SARS-CoV-2 were recruited between November and December 2020. Patients were excluded if they were in immediate need of intubation or mechanical ventilation, had advanced pulmonary or cardiac disease, current malignancy, previous thromboembolic disease, current pregnancy, or were unable to tolerate the study protocol, which required spontaneous breathing of ambient air for several minutes. Data were collected within 24 h of presentation to hospital and again 55±10 days after discharge in early recovery (n=17). 13 patients did not return for follow-up. A subset of the acute data has been reported previously (41).

13 healthy volunteers, negative for SARS-CoV-2 on PCR testing and with normal pulmonary function (Vyaire-Vyntus Spiro PC-spirometer with SentrySuite Software; Vyaire, Mettawa, IL, USA), were recruited from hospital staff as methodological controls.

All participants gave written informed consent, and the study protocol was approved by the Swedish Ethical Review Authority (diary number 2020-02966). Subject characteristics are shown in Table 1.

Clinical data Anthropometric and demographic data were collected, along with body temperature for the acutely ill. Pathology test results, pharmacological interventions, respiratory support (including oxygen therapy) and duration of hospitalization data were also collected.

Study protocol Subjects were studied in a semirecumbent position, wearing a noseclip and breathing ambient air via a mouthpiece with an antivirus filter (MicroGard II; Vyaire Medical, Hoechberg, Germany; apparatus deadspace 75 mL) attached to an inspired/expired gas measurement system (Oxycon Pro; Vyaire Medical). Collected data included PO₂, PCO₂ and respiratory gas flow at high sampling frequency (100 Hz). After a few minutes of adaptation, participants maintained steady-state breathing (end-tidal PCO₂ within ±2 mmHg across several breaths) at a (metronome-facilitated) frequency and tidal volume of their choice. Data were recorded over a steady-state 3-5 min period, during which radial arterial blood was collected over several breaths and then processed immediately (ABL800 Flex Plus; Radiometer Medical, Bronshoj, Denmark) to obtain values for arterial PO₂ (PaO₂) and PCO₂ (PaCO₂). For the acute patient studies, arterial blood gas values were expressed at body temperature (46). Recovered patients and healthy subjects were assumed to have a body temperature of 37° C.

Exhaled gas analysis Three separate breaths, preceding the arterial blood sample by 35±10 s, were selected, independently analysed and resulting parameters averaged. Following alignment of gas and volume signals, PO₂ and PCO₂ values for each breath were plotted as a function of expired volume and a linear least-squares fit applied to the alveolar plateau (phase III; see FIG. 1). Mean alveolar gas values, PAO₂ and PACO₂ (45), at the mid-point (by volume) of the expired breath were determined from the fitted lines. Exhaled gas measurements in the acute patient studies were corrected for water vapour pressure at body temperature using Antoine's formula (47). Alveolar-arterial partial pressure differences for both O₂ and CO₂ were then calculated as PAO₂−PaO₂ (PA-aO₂) and PaCO₂−PACO₂ (Pa-ACO₂).

Computational analysis The RILEY and COURNAND (48) three-compartment lung model was used to calculate shunt and alveolar deadspace from PA-aO₂ and Pa-ACO₂. In this model, the lung is considered to have an intrapulmonary shunt compartment with a \dot{V}A/\dot{Q} of zero, also encompassing regions of very low \dot{V}_{A}/\dot{Q}, and an alveolar deadspace compartment with \dot{V}A/\dot{Q} of infinity, also encompassing regions of very high \dot{V}A/\dot{Q}. The remainder of the lung is assumed to be normal, with a \dot{V}A/\dot{Q} ratio given by non-deadspace ventilation divided by non-shunt blood flow. A critical difference exists between the Riley and Cournand model and the current approach: Riley and Cournand used the venous admixture equation for oxygen to calculate shunt and the Bohr equation for carbon dioxide to calculate deadspace. However, shunt may increase arterial PCO₂ and contribute to calculated alveolar deadspace, while deadspace (without compensatory hyperventilation) lowers arterial PO₂ and contributes to calculated shunt. Our approach (45) recognises this complexity and resolves it, still within the three-compartment framework, by determining the values of shunt and deadspace that, present together, predict the measured arterial and expired alveolar PO₂ and PCO₂ values, and hence their partial pressure differences. In addition, the Bohr equation usually includes anatomical deadspace, which commonly dominates total deadspace numbers. Our approach eliminates anatomical deadspace by using mean alveolar partial pressures as in FIG. 1 and not those of mixed exhaled gas.

Using the measured \dot{V}CO₂ (respiratory frequency×volume CO₂ per breath), PAO2, fractional alveolar oxygen concentration (FAO₂), PACO₂ and fractional alveolar carbon dioxide concentration (FCO₂), the following were calculated: 1) total expired alveolar ventilation \dot{V}A=\dot{V}CO₂/FACO₂, 2) inspired alveolar ventilation \dot{V}_{I}=\dot{V}A×((1−FAO₂−FACO₂)/(1−FIO₂)) and 3) \dot{V}O₂=\dot{V}_{I}×FIO₂-\dot{V}A×FAO₂, where FIO₂ is the inspiratory oxygen fraction.

Cardiac output (\dot{Q}_{T}) was then estimated as \dot{Q}_{T}=\dot{V}\dot{Q}₂+5, with both \dot{Q}_{T} and \dot{V}O₂ expressed in L-min−1 (49-50). The oxygen tension at which haemoglobin is 50% saturated (HbP50) was assumed to be 26.8 mmHg. Using these values and each patient's own data for \dot{V}O₂, \dot{V}CO₂, \dot{V}_{A}, \dot{Q}_{T}, base excess, haemoglobin concentration and body temperature, the algorithm first published in 1969 by West (51) was used to estimate the values of intrapulmonary shunt and alveolar deadspace that would result in the measured PaO₂ and PaCO₂ in each individual to within 0.2 mmHg.

Intrapulmonary shunt was expressed as the percentage of pulmonary blood flow perfusing unventilated regions (shunt %), while alveolar deadspace was expressed as the percentage of alveolar ventilation associated with unperfused regions (alveolar deadspace %). Based on published measurements using the multiple inert gas elimination technique in normal subjects (52), the 95% upper confidence limit for physiological shunt is 5% and for alveolar deadspace is 10%. Further methodological details can be found elsewhere (45).

TABLE 1
Subject characteristics

| | Acute | Recovered | Healthy |
|---|---|---|---|
| | (n = 30) | (n = 17) | (n = 13) |
| Age (years) | 49.9 ± 13.5 | 50.6 ± 12.0 | 51.1 ± 17.0 |
| | (23-78) | (51-78) | (34-68) |
| Sex | | | |
| Female | 8 | 6 | 5 |
| Male | 22 | 11 | 8 |
| Weight (kg) | 87.5 ± 17.0 | 87.5 ± 18.2 | 80.2 ± 26.1 |
| | (46-113) | (46-110) | (58-110) |
| Height (cm) | 177.0 ± 11.3 | 176.8 ± 13.2 | 177.2 ± 19.5 |
| | (150-195) | (150-195) | (159-198) |
| BMI (kg · m⁻²) | 27.8 ± 4.3 | 27.9 ± 4.7 | 25.5 ± 6.0 |
| | (20.2-38.5) | (20.4-38.5) | (19.6-31.5) |
| Body temperature | 38.0 ± 1.0 | 37.0^{#} | 37.0^{#} |
| (° C.) | (36.5-40.0) | | |

Data are presented as mean ± SD (range) or n.
^{#}assumed.

Statistical analysis This was an observational study and a sample size was not calculated. Individual data were pooled and reported as group mean with standard deviation or interquartile range. Comparisons were made using paired t-tests. Relationships between variables were examined using Spearman's rank correlation coefficients. p<0.05 was considered significant.

Acute COVID-19 patient data All patients had moderate disease and were studied at hospital presentation and within 3-15 days of symptom onset. 24 were admitted (mean 4.0±2.8 days, range 1-12 days); of these, 18 required supplemental oxygen and one was subsequently admitted to the intensive care unit. There were no deaths.

Pharmacological interventions 22 patients received prophylactic low-molecular-weight heparin (LMWH), 12 of these patients <24 h before the study (tinzaparin 4500 U per day, subcutaneously), while therapeutic LMWH (tinzaparin 175 U-kg−1 twice daily, daily, s.c.) was administered to one patient. Post-study, 14 patients received corticosteroids (betamethasone 6 mg orally), three received remdesivir and convalescent plasma was administered to two patients.

Pathology findings C-reactive protein levels were elevated in all patients, while almost 40% had elevated levels for D-dimer (Table 2).

TABLE 2
Pathology data at the time of acute COVID-19 infection

| | | | Normal | Abnormal |
|---|---|---|---|---|
| Pathology | Mean ± SD | Range | range | (n (%)) |
| Haemoglobin | 139.5 ± 11.7 | 110-165 | Female: 117- | 2 (6.8) |
| (g · L⁻¹) | | | 153; male: | |
| (n = 29) | | | 134-170 | |
| D-dimer | 0.8 ± 0.6 | 0.3-3.2 | Female: <0.54; | 11 (39.2) |
| (mg · L⁻¹) | | | male: <0.50 | |
| (n = 28) | | | | |
| C-reactive protein | 79.1 ± 65.7 | 3-256 | <3 | 30 (100) |
| (mg · L⁻¹) | | | | |
| (n = 30) | | | | |

Arterial blood gas, oxyhaemoglobin saturation and exhaled alveolar gas data Arterial blood gas data reflected an acute respiratory alkalosis with hypoxaemia (Table 3). PAO₂ values were all >102 mmHg, PACO₂ values were all <38 mmHg and arterial oxygen saturation (SaO₂) values were all >89%.

TABLE 3
Arterial blood gases and exhaled alveolar gas measurements

| | Acute | Recovered | Healthy |
|---|---|---|---|
| | (n = 30)^{#} | (n = 17) | (n = 13) |
| pH | 7.48 ± 0.04 | 7.44 ± 0.03 | 7.43 ± 0.02 |
| | (7.42-7.60) | (7.39-7.51) | (7.41-7.49) |
| P_{aCO2} (mmHg) | 34.8 ± 4.2 | 34.4 ± 3.7 | 37.0 ± 2.8 |
| | (26.5-43.0) | (26.7-40.6) | (31.4-40.8) |
| P_{aO2} (mmHg) | 68.3 ± 12.6 | 100.6 ± 13.6 | 105.0 ± 8.3 |
| | (52.3-105.8) | (84.0-131.3) | (94.5-120.0) |
| P_{AO2} (mmHg) | 113.3 ± 5.7 | 113.6 ± 6.3 | 119.5 ± 5.1 |
| | (102.2-122.3) | (100.2-125.3) | (112.7-127.3) |
| P_{ACO2} (mmHg) | 30.3 ± 4.0 | 31.4 ± 3.1 | 35.2 ± 2.9 |
| | (23.4-37.3) | (24.9-38.7) | (29.1-39.9) |
| P_{A-aO2} (mmHg) | 41.4 ± 16.3 | 13.0 ± 9.7 | 6.5 ± 6.9 |
| | (−3.5-69.3) | (−6.0-30.2) | (−5.8-21.4) |
| P_{a-ACO2} (mmHg) | 6.0 ± 4.2 | 3.0 ± 2.3 | 1.8 ± 1.9 |
| | (−2.3-13.4) | (−2.2-8.9) | (−1.4-6.0) |
| S_{aO2} (%) | 94.3 ± 0.2 | 98.7 ± 0.5 | 98.9 ± 0.2 |
| | (89.4-98.7) | (97.7-99.5) | (98.4-99.3) |

Data are presented as mean ± SD (range).
^{#}all acute patient gas partial pressures are expressed at body temperature.
P_{aCO2}: arterial partial pressure of carbon dioxide; P_{aO2}: arterial partial pressure of oxygen; P_{AO2}: alveolar partial pressure of oxygen; P_{ACO2}: alveolar partial pressure of carbon dioxide; P_{A-aO2}: alveolar-arterial partial pressure difference for oxygen; P_{a-ACO2}: alveolar-arterial partial pressure difference for carbon dioxide; S_{aO2}: arterial oxygen saturation.

Alveolar-arterial differences There was wide interpatient variance in PA−aO₂ and Pa−ACO₂ (table 3 and FIG. 2a). Any negative values (expected from experimental noise) were considered to be zero when calculating shunt and deadspace.

Intrapulmonary shunt and alveolar deadspace Values for intrapulmonary shunt and alveolar deadspace varied considerably between patients (table 4 and FIG. 2b); however, importantly, there was no significant correlation between the shunt and deadspace values (p=0.27). Significant positive correlations were detected between shunt and both D-dimer (r=0.36, p=0.03) and CRP (r=0.42, p=0.01), but not for alveolar deadspace (p>0.15).

TABLE 4
Shunt and alveolar deadspace in acute COVID-19 patients
(including the subgroup of 17 patients studied later
in recovery), recovered patients and healthy subjects.

| | Acute | Acute^{#} | Recovered | Healthy |
|---|---|---|---|---|
| | (n = 30) | (n = 17) | (n = 17) | (n = 13) |
| Shunt (%) | 10.4 ± 5.4 | 11.7 ± 5.1 | 2.4 ± 1.9** | 1.2 ± 1.1 |
| | (0-22.0) | (4.1-22.0) | (0-6.1) | (0-4.1) |
| Alveolar | 14.9 ± 9.6 | 14.4 ± 9.7 | 8.6 ± 5.5* | 4.7 ± 4.3 |
| deadspace (%) | (0-32.3) | (0-29.7) | (0-22.4) | (0-11.8) |

Data are presented as mean ± SD (range).
^{#}subgroup studied in recovery.
*p < 0.05;
**p < 0.0001 (compared with acute subgroup).

Overall, 23 patients had elevated shunt (defined as >5%), while 22 patients had elevated alveolar deadspace (defined as >10%). Simultaneously increased shunt and PGP-deadspace were present in 19 patients, four patients had elevated shunt but normal alveolar deadspace and three had elevated alveolar deadspace but normal shunt (FIG. 2b).

Healthy subject data Healthy subjects were all normoxaemic with normal acid-base status (see table 3). No healthy subject had a shunt value >5% (table 4 and FIG. 2b); two had slightly elevated values for deadspace (table 4 and FIG. 2b). Overall, shunt and deadspace values from the healthy subjects conformed to the previously established upper limits of normal (52).

Transition from acute illness to recovery Shunt and alveolar deadspace fell significantly following recovery (FIGS. 3 and 4). However, shunt was slightly elevated in two patients, while five patients (29.4%) had elevated deadspace. There was no correlation with duration after recovery (both p>0.15).

Using simultaneous measurements of arterial and exhaled oxygen and carbon dioxide tensions, interpreted using a three-compartment computational lung model, a bedside methodology was developed for quantifying if there is both intrapulmonary shunt and alveolar deadspace (45), and then applied it here in the highly infectious disease setting of COVID-19 pneumonitis. Measurements in a healthy subject cohort (technical controls) conformed to historical normal limits previously established by more sophisticated techniques (52).

### Pulmonary Gas Exchange in Early COVID-19 Pneumonitis

The present technology shows that in early acute COVID-19 pneumonitis intrapulmonary shunt is elevated for most patients; however, many also have elevated alveolar deadspace. Furthermore, intrapulmonary shunt and deadspace values were not correlated in moderate COVID-19 pneumonitis. Hence, deadspace cannot be predicted from the magnitude of shunt and vice versa. This suggests that in early SARS-CoV-2 pulmonary infection two pathophysiologically distinct process, either separately or together, are in play: 1) patchy alveolar filling/oedema/atelectasis (pneumonia) resulting in lung regions with low or zero \dot{V}A/\dot{Q}(i.e. shunt) and 2) patchy pulmonary vascular occlusion (emboli) resulting in high \dot{V}A/\dot{Q} regions (i.e. alveolar deadspace). However, the relative contribution of these two processes is highly variable from patient to patient. This is similar to findings in intubated patients with severe acute respiratory distress syndrome, where hypoxaemia is due to increased intrapulmonary shunt and increased deadspace (53). This is the first time that this has been demonstrated physiologically in mild/moderate COVID pneumonitis.

While intrapulmonary shunt is perhaps an expected contributor to hypoxaemia in a pulmonary viral infection (53), previous authors have suggested that for COVID-19 patients shunt values can be so elevated as to be considered “excessive” for the degree of lung injury present (37). This observation has led to the suggestion that SARS-CoV-2 may specifically impair hypoxic pulmonary vasoconstriction, thus preventing hypoxic pulmonary vasoconstriction-driven restriction of pulmonary capillary blood flow to lung regions with poor or no ventilation, increasing shunt (37, 54). Whether intrapulmonary shunt values detected in the present study are “excessive” remains undetermined.

A feature of the present technology was the high deadspace values occurring in 73% of patients, with 63% having both elevated intrapulmonary shunt and deadspace and 10% having elevated deadspace with no evidence of shunt. Increased alveolar deadspace is a consequence of ventilation of underperfused alveoli, which may be consistent with reported diffuse and widespread small pulmonary arterial obstruction from thrombus formation, as detected in pulmonary pathological specimens from deceased COVID-19 patients (42, 55). This interpretation is further supported by the absence of large vessel emboli on contrast computed tomography imaging (performed for clinical purposes) in a subgroup (n=18) of the present patient cohort, although there was no correlation between deadspace and D-dimer.

There is extensive pathological evidence of pulmonary microvascular involvement in COVID-19 pneumonia (55). Histopathology of patients who died from COVID-19, compared with those who died from influenza, showed microvascular changes present in COVID-19 infected lungs that were not present with influenza, including endothelial injury, alveolar capillary microthrombi and new vessel growth (42). In a study which correlated radiological findings with histopathological inflammation in eight patients who died of COVID-19, there was evidence of vascular damage and thrombosis in regions without concurrent airspace involvement (56). It has been proposed that the vascular injury may precede the development of frank pneumonia and alveolar filling (59).

High \dot{V}A/\dot{Q} regions (deadspace) may also be a consequence of redistribution of ventilation from unventilated alveoli (shunt) resulting in relative overventilation of normally perfused alveoli. Our analysis adjusts for any effect of intrapulmonary shunt on Pa-ACO₂, so that redistribution of ventilation from obstructed ventilation zones is unlikely to contribute significantly to our measured deadspace values.

Pulmonary gas exchange in early recovery from COVID-19 pneumonitis In recovery, intrapulmonary shunt decreased in all patients and values were within normal expected limits for all but two patients (FIGS. 3 and 4). Consequently, ventilation was restored to lung zones previously identified as having no or very low alveolar ventilation. Deadspace also decreased in most patients to within, or close to, normal expected limits (FIGS. 3 and 4). However, in five patients, deadspace was >10%, ranging from 10.7% to 22.4%. Consequently, for most patients perfusion was restored to lung zones previously identified as having no or very low perfusion. However, for ˜30% of those studied in recovery (FIGS. 3d and 4), persistent, or even increasing, deadspace values suggest persistent or evolving damage to the pulmonary vasculature. This may be a consequence of pre-morbid disease (e.g. COPD or interstitial lung disease). However, if a consequence of COVID-19 infection, this finding is of particular interest, since recently published data suggest that COVID-19 infection poses increased risk for deep vein thrombosis, pulmonary embolism and bleeding episodes, at 3, 6 and 2 months, respectively, after an acute infection (58).

Clinical implications The finding that there is likely both acute and chronic pulmonary microvascular disease in COVID-19 pneumonitis has implications for the clinical management of patients, both in the acute and recovery phases of the disease process. For example, anticoagulation with LMWH has had mixed outcomes, with benefit in moderate disease but no real improvement found in severe disease (59-60). Perhaps a clearer picture might emerge if outcomes were stratified by presence or absence of high \dot{V}A/\dot{Q} regions. Targeted therapeutic approaches may then be deployed for patients with increased deadspace, who may need anticoagulation, versus those with shunt but low deadspace, where anticoagulants may, in theory, have risk without major benefits. Application of this methodology could be used in patients with persistent COVID-19 symptoms to better delineate pulmonary pathology.

This study was performed in a small sample of patients with early and mild/moderate disease, early in the pandemic, when few therapeutic interventions were available, and was restricted to those able to tolerate breathing room air. Also, recovery data were collected at only one time-point without follow-up data on all patients. Consequently, it may not be generalisable to more severe disease. Other (pre-existing) comorbidities such as anaemia may have impacted on our findings, although there was no clinical evidence of these abnormalities. The logistical limitations imposed by an acute airborne infection meant that we were unable to use more sophisticated techniques such as multiple inert gas elimination (61) or imaging tools (62).

We estimated cardiac output and HbP50, required for the calculation of intrapulmonary shunt and deadspace. Cardiac output was determined from its well-documented relationship to \dot{V}O₂ (49-50), which was measured; HbP50 was taken to be 26.8 mmHg for all participants. Sensitivity analysis for these two uncertain variables shows a minor effect over a wide range of determined intrapulmonary shunt values, with no effect on deadspace (45).

Conclusions Our study shows that in early mild/moderate COVID-19 pneumonitis, there is both increased intrapulmonary shunt and increased alveolar deadspace, with marked interpatient variability and lack of correlation. These findings suggest that alveolar filling, resulting in little or no ventilation to some alveoli, and pulmonary microvascular compromise, resulting in little or no blood flow to other alveoli, are both present to varying, but separate, degrees in the early phases of mild/moderate COVID-19 pneumonitis. For essentially all patients, shunt resolved in early recovery; however, for ˜30% of patients, elevated deadspace persisted, at least in the early recovery phase. Characterising individual patient pulmonary pathophysiological responses may help inform more personalised approaches to effective treatments in both the acute and recovery phases of infection with SARS-CoV-2.

### Example 6: Pulmonary Shunt and Deadspace Post COVID-19

COVID-19 is a disease caused by the SARS CoV-2 virus (63). Acute severe COVID-19 is characterised by acute pneumonitis, pulmonary vascular endothelialitis and microemboli leading to arterial hypoxemia associated with increased intra-pulmonary shunt and alveolar deadspace (64-65).

Longer term respiratory sequelae, including persistent dyspnea, impaired pulmonary function and abnormal radiological imaging, are reported in 20-30% of patients following recovery from acute SARS CoV-2 infection (66-67). Post COVID-19, some patients presenting with unexplained persistent dyspnea are reported to be hyperventilating based on questionnaire, arterial hypocapnia and cardiopulmonary exercise response (68). A recent meta-analysis (69) found that ˜45% of post COVID-19 patients have radiologic evidence of pulmonary fibrosis. Resultant effects on pulmonary gas exchange remain to be established, although some recent MRI studies using hyperpolarized 129Xe MRI (70-71), demonstrate abnormal transfer of Xe gas from the tissue or the parenchyma even in the presence of near normal lung imaging. This may be due to regional decreases in perfusion leading to an increase in alveolar deadspace.

Using a novel technique, based on measurement of exhaled oxygen and carbon dioxide combined with arterial blood gas analysis (72), we recently reported impaired pulmonary gas exchange (based on multiple inert gas elimination technique values (MIGET) where intra-pulmonary shunt exceeding the 95% upper confidence limit of 5% in normal subjects and increased alveolar deadspace exceeding the 95% upper confidence limit of 10% in normal subjects (73)) in 30 patients studied during the early stages of mild-moderate acute COVID-19 and then, in a sub-group (n=17), of these patients studied at up to 72 days post-acute infection (65). Intrapulmonary shunt was defined as an alveolar ventilation/perfusion ratio (\dot{V}A/\dot{Q}) of 0, also encompassing regions of very low \dot{V}A/\dot{Q}, alveolar deadspace was defined as a \dot{V}A/\dot{Q} of infinity, also encompassing regions of very high \dot{V}A/\dot{Q}.

For most patients, both shunt and alveolar deadspace were abnormally high at the time of the acute study and reduced at the time of the follow-up study; however, shunt at follow-up remained marginally above normal for 2 patients while deadspace was abnormal for 5 patients (i.e. ˜40% of this small cohort had persistent functional pulmonary gas exchange abnormality). Whether these patients would go on to recover in the longer term, or if there is more permanent lung damage (e.g. fibrosis) sufficient to affect gas exchange, is not known. Nor is it understood why gas exchange impairment persists into the post COVID-19 recovery period for some patients and not others.

In addition, the prevalence, level and underlying cause of reported hyperventilation in the post COVID-19 population are not clear. In the acute phase of the disease, we have previously shown 50% of these patients had alveolar ventilation greater than required for normocapnia (relative alveolar ventilation), and also, perhaps more importantly greater than expected for the level of hypoxemia (74). Whether this finding persists post COVID-19 is unknown.

In this study we hypothesized that post COVID-19 patients with a history of more severe acute illness would have greater and longer-lasting shunt and alveolar deadspace values during recovery than patients who had experienced a less severe acute illness. We also hypothesized that increased alveolar ventilation, if present post COVID-19, would be related to the degree of shunt and/or deadspace present. Accordingly, we measured intra-pulmonary shunt alveolar deadspace, and relative alveolar ventilation in a cohort of post COVID-19 patients (n=59), studied at up to 403 days post-acute illness and also determined risk factors for ongoing pulmonary gas exchange impairment.

### Subjects

Post COVID-19 Patients In this cross-sectional, observational study, 59 patients (never-vaccinated for COVID-19) previously diagnosed with SARS-CoV-2 infection by PCR positive nasal swab between March 2020 and January 2021 at Westmead Hospital, Sydney, Australia were recruited from a routine follow-up clinic and studied between 15 and 403 days (152.3±85.4 (mean±SD)) after their initial positive PCR test. None of these subjects were part of our previous study (65, 74).

Recruitment was open to all post COVID-19 patients irrespective of the severity of acute illness, whether managed at home or in hospital, and whether or not they were experiencing any ongoing symptoms. There were no other inclusion or exclusion criteria. Of the 59 participants, 44 had not required admission for their acute disease episode, however 15 patients had been admitted, with 7 requiring supplemental oxygen (5 via high flow nasal cannulae), and 5 requiring intubation and mechanical ventilation.

All patients were studied between June 2020 and May 2021, following protocol approval by the western Sydney local health district human research ethics committee, (HREC: 2020/ETH01610) and all gave written informed consent. A data use agreement allowed sharing of data amongst the co-authors.

Acute COVID-19 Severity (NIH category) Based on the medical record, patients were categorised using NIH COVID-19 severity categories (75), (NIH-1: asymptomatic; NIH-2: any symptom of COVID-19 but no dyspnea, or abnormal chest imaging; NIH-3: evidence of a lower respiratory disease during clinical assessment or imaging, however, oxygen saturations >94%; NIH-4: either oxygen saturation <94%, PaO₂/FIO₂<300 mmHg, respiratory rate >30 breaths/min, or lung infiltrates >50%; NIH-5: respiratory failure, septic shock and/or multiple organ dysfunction).

Ancillary Data Patient characteristics recorded included anthropometrics, smoking history, previous non-COVID-19 diagnoses such as hypertension, diabetes, lung or heart disease, date of SARS-CoV-2 PCR positivity, and whether hospitalized for COVID-19 (including oxygen administration or intubation). Respiratory symptoms, at the time of follow-up, including cough and breathlessness, were recorded.

Pulmonary Shunt and Alveolar Deadspace The theory underlying the “bedside methodology” we used to quantify intra-pulmonary shunt and alveolar deadspace using exhaled and arterial blood gas analyses has been previously presented in detail (72) and has also been the subject of recent editorial review (76).

Methodology For the present study, the methodology protocol was similar to that outlined in our previous publication (65). In brief, seated subjects wore a nose clip while breathing ambient air on a mouthpiece with attached pneumotachometer and gas sampling connection, allowing continuous monitoring of inhaled and exhaled oxygen, carbon dioxide and airflow (Medgraphics Ultima PFX, MGC Diagnostics, Minnesota, USA). Data were collected at 100 Hz for about 2 minutes and stored as raw time-based voltage signals for later determination of mean alveolar PO₂ and PCO₂. Immediately afterwards, during steady state breathing (i.e., end-tidal CO₂ changing by <2 mmHg over 4 consecutive breaths), arterial blood (˜1 ml) was collected over 2-3 breaths from a radial artery puncture using a heparinised syringe (Radiometer PIC070 with 25-gauge needle) and analyzed within 3 minutes (Radiometer ABL800 FLEX gas analyser, maintained at 37° C.).

Arterial Blood Gas Analysis, Arterial blood pH, PaCO₂ and PaO₂ were recorded, and assumed to be at₃₇° C.

Exhaled Gas Analysis Time-based voltage data were exported into MATLAB. Imported data were adjusted for lag time due to: a) the transport delay time (time from flow reversal to start of the fall in CO₂ (or rise in O₂) towards inspired at the end of a breath, and b) the time it takes from that start of end-tidal change towards inspired to reach the half-way mark (ie, to the average of end-tidal and inspired) (77).

Voltage data for O₂ and CO₂ (100 Hz) were converted to partial pressures using calibration curves. Minor differences in day-to-day calibration were accounted for by rescaling so that the inspired and end-tidal values for both gases matched corresponding partial pressures available from the Medgraphics outputs collected at the time of arterial blood sampling.

Calculation of alveolar partial pressures and alveolar-arterial partial pressure differences Three separate breaths were selected, independently analysed, and resulting parameters averaged. Following alignment of gas and volume signals, PO₂ and PCO₂ values for each breath were plotted as a function of expired volume, and a linear least-squares fit applied to the alveolar plateau. Mean alveolar gas values, PAO₂ and PACO₂ (72) at the mid-point (by volume) of the expired breath were determined from the fitted lines. Alveolar-arterial partial pressure differences for both O₂ and CO₂ were then calculated as PAO₂−PaO₂ (AaPO2) and PaCO2−PACO₂ (aAPCO₂).

Shunt and Alveolar Deadspace Shunt and alveolar deadspace were derived from the alveolar arterial differences for the two gases as has been previously described in detail (72). Using the 3-compartment model of Riley and Cournand (78), in which the lung is considered to consist of: 1) an intrapulmonary shunt compartment with an alveolar ventilation/perfusion ratio (\dot{V}A/\dot{Q}) of 0, also encompassing regions of very low \dot{V}A/\dot{Q}, 2) an alveolar deadspace compartment with \dot{V}A/\dot{Q} of infinity, also encompassing regions of very high \dot{V}A/\dot{Q}, and 3) the remainder of the lung, with a \dot{V}A/\dot{Q}ratio given by non-deadspace ventilation divided by non-shunt blood flow. Briefly the analysis runs as follows. Firstly, both AaPO₂ and aAPCO₂ are each affected by both intrapulmonary shunt (and low \dot{V}A/\dot{Q}regions) but also by alveolar deadspace (and high \dot{V}A/\dot{Q}regions), as explained previously (72) (see FIG. 1). The analysis uses both AaPO2 and aAPCO₂ to simultaneously determine the amount of intra-pulmonary shunt and alveolar deadspace that together would uniquely yield the measured alveolar-arterial differences for both gases. This is achieved using the computer program published by West in 1969 (79). Firstly, the multi-compartment log-normal \dot{V}A/\dot{Q}distribution model is replaced with the three compartment model (78). Following this, rather than using West's computer model in the intended manner to determine arterial and expired pO₂ and pCO₂, and hence the arterial-alveolar differences, the program is run in reverse, allowing the intrapulmonary shunt and alveolar deadspace that would result in the measured alveolar-arterial differences to be determined. Several other variables are required in addition to the measured alveolar-arterial differences, and these include body temperature (assumed to be 37° C.), acid-base status and hemoglobin (both measured on the arterial blood gas), ventilation, \dot{V}O₂, \dot{V}CO₂, FIO₂ (all measured during expired gas analysis). Hemoglobin P50 was assumed to be 27 mmHg, and the cardiac output was estimated from the empirical relationship with oxygen uptake as 5×\dot{V}O₂+5 (80), which are reasonable assumptions that do not greatly impact on shunt estimation (72). Importantly, the deadspace compartment reflects only alveolar deadspace, being based on the differences between arterial and mean alveolar PO₂ and PCO₂. Thus, anatomic deadspace is excluded.

Methodological Validation As a validation of methodology used in the present study, we measured intra-pulmonary shunt and alveolar deadspace in 7 healthy adults (no history of COVID-19; 3 males, age 32.9±14.7 years [mean±SD], body mass index (BMI) 24.6±2.3 kg/m2).

Comparison with Traditional Measurements of Gas Exchange Bohr-Enghoff deadspace (%) was calculated for each subject using the measured mixed expired CO₂ (PECO₂) and PaCO2, Bohr-Enghoff Deadspace %=(PaCO₂−PECO₂)×100 and PaCO2 plotted against measured alveolar deadspace.

In addition, AaO2 was calculated using PaCO2 PAO₂=PIO₂−PaCO2/R+PaCO2×FIO₂× (1−R)/R using R, PIO₂ and FIO₂ measured in the exhaled gases.

Relative Ventilation We also assessed each patient's level of alveolar ventilation by calculating the term 40/PACO₂, designated VArel, which indicates alveolar ventilation relative to that which would be present had PACO₂ been 40 mmHg. VArel was plotted against PaO₂ (74), PaCO2, shunt and alveolar deadspace. For comparison, historical healthy young subject data (81-83) were used.

Statistical Analysis Continuous data were pooled and reported as mean and standard deviation for parametric data or median and inter-quartile range (IQR) for non-parametric data. Categorical data were summarised by proportions and counts. NIH severity score of 1 or 2 were grouped and classified as ‘low-medium severity’ sub-group, while NIH scores of 3, 4, or 5, were grouped and classified as ‘severe-critical severity’ sub-group. As previously, 95% confidence limits for intra-pulmonary shunt were defined as <5% and normal alveolar deadspace as <10%, based on values derived from healthy subjects using the multiple inert gas elimination technique (73) (79).

Univariate comparisons were tested via Mann-Whitney U tests. Correlations were examined using Spearman's rank correlation co-efficient.

Univariate and multivariate gaussian regression models were used to examine relationships between gas exchange parameters (shunt and alveolar deadspace) and acute COVID-19 illness severity (NIH category), time since acute illness, age, BMI, and co-morbid diabetes/hypertension. Statistical analyses were completed using Prism 9 (Version 9.5.1, GraphPad Software LLC, Boston, MA, USA) or Stata SE Version 14.2 (Stata Corp LLC, College Station, TX, USA).

Patient Data Patients were between 19 and 81 years of age (average 50.4±16.1 years) with a BMI of 30.7±8.4 kg/m2. We studied 33 women and 26 men. Fourteen (23.7%) had a history of any smoking, 3 (5.1%) had a more than 10 pack year history, 2 (3.4%) had a diagnosis of asthma, 9 (15.3%) had a diagnosis of diabetes, 12 (20.3%) had hypertension and 3 (5.2%) known cardiovascular disease. Forty-four (74.6%) had previous mild-moderate COVID-19 (6 NIH-1, 38 NIH-2), while 15 (25.4%) had previous severe-critical COVID-19 (3 NIH-3, 7 NIH-4, 5 NIH-5). Patients were studied between 15-403, a median of 127 (95-186) days after 1^{st} SARS-CoV-2 PCR positive test.

Respiratory Symptoms Respiratory symptoms were documented in 51/59 patients at follow up. Of these 9 (18%) patients complained of a cough, 23 (45%) patients complained of breathlessness, 5 (10%) patients complained of wheeze. Overall, 25 (49%) of patients had at least one respiratory symptom (cough, breathlessness and/or wheeze).

Arterial Blood Gas and Exhaled Gas Data. FIG. 2 shows group arterial blood gas and exhaled gas data grouped by NIH severity. Patients with a history of Low-Medium COVID-19 severity has a significantly higher PaO₂ (94.0 (88.7-101.8) mmHg, compared with those with Severe-Critical COVID-19 severity (87.1 (83.3-91.0) mmHg: P<0.005).

The average hemoglobin for female patients (n=26) was 131 (123-138) g/I and the average for male subjects was 145 (135-155) g/I.

Shunt and Alveolar Deadspace FIG. 3 shows individual data for alveolar-arterial differences, and shunt and alveolar deadspace values. Median AaPO₂ values were significantly higher in NIH category 3-5 compared to NIH category 1-2 subgroup (24.7 (18.6-33.9) mmHg vs 16.9 (11.0-25.8) mmHg; p<0.008). Median shunt values were also significantly higher in the NIH category 3-5 subgroup than in the NIH category 1-2 group (6.5 (˜0.2-13.2) % vs 3.5(−0.1-7.0) %, p<0.01); however, there was no significant difference in aAPCO₂ or alveolar deadspace between the subgroups.

FIG. 4 shows relationships between alveolar-arterial differences, and shunt and alveolar deadspace values for methodological controls and for post COVID-19 patients. All of the methodological control data lies within, or close to, the 95% confidence levels, indicating technical adequacy of the AaPO₂ and aAPCO₂ measurement. Only 8 of the 59 patients (13.5%) had shunt values <5% and alveolar deadspace values <10%. One (1.6%) patient had increased shunt but normal alveolar deadspace, while 21 (35.5%) had both increased shunt and alveolar deadspace, and 29 (49.2%) had increased deadspace only. Thus, 37% had elevated shunt and 86% had elevated deadspace. There was no correlation between shunt and alveolar deadspace (p=0.07). There were no differences between shunt and alveolar deadspace between those with or without shortness of breath (p=0.4 and 0.49 respectively).

Comparison with Bohr-Enghoff Deadspace and Calculations using the Alveolar Gas Equation. Bohr-Enghoff deadspace calculated using either measured mixed expired CO₂, and alveolar-arterial PaO₂ calculated using the alveolar gas equation plotted against alveolar deadspace and measured AaPO₂ are shown in FIG. 5. This plot shows that Bohr-Enghoff deadspace measurements are much greater than the measured alveolar deadspace measurement and AaO₂ gradient calculation underestimates the true AaO₂ gradient. There were significant linear relationships between Bohr-Enghoff deadspace and alveolar deadspace (R2 0.65, p<0.0001) and calculated and measured AaO₂ gradient (R2 0.8, p<0.0001).

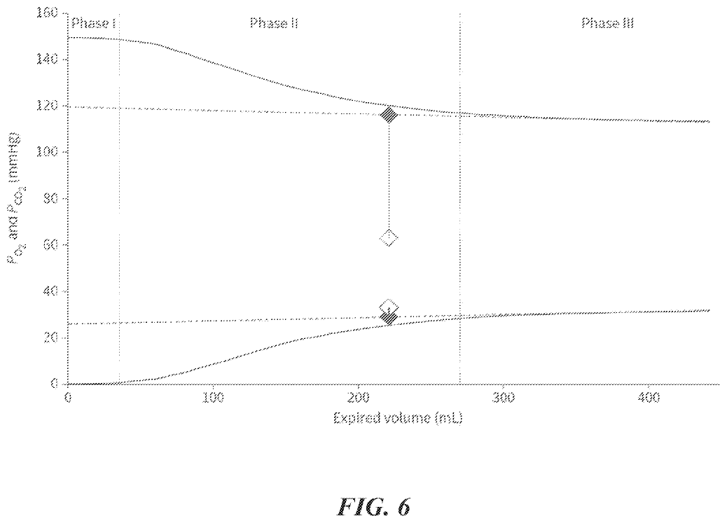

Acute Illness Severity Category FIG. 6 presents shunt and deadspace for post COVID-19 patients plotted against time since infection grouped by NIH severity category. There were no significant differences between patient sub-groups in time from 1 st positive PCR, age, height, weight, smoking prevalence or previous diagnosis of asthma (all p>0.05). Patients in the severe-critical category were significantly more obese (BMI: 34.3(27.8-38.3) kg/m2 vs 28.0 (25.3-31.1) kg/m2; p<0.01), and more likely to have a previous diagnosis of diabetes (8 (NIH 3-5) vs 1 (NIH 1-2)) or hypertension (8 (NIH 3-5) vs 4 (NIH 1-2); both p<0.01).

Outcomes from Regression Models For shunt, there were significant univariate relationships with: BMI (0.11 (0.02-0.21) % per kg/m2, (95% confidence interval, p=0.0²), age (0.05 (0.00-0.11) % per year, p=0.04), and acute illness NIH severity category (3.1 (1.34-4.86) % per category, p<0.01). However, in the multivariate models only NIH severity category was a significant predictor for shunt (2.69% per category (0.82-4.57), p<0.01). For alveolar deadspace, there was a small, but significant, univariate relationship with age (1.6 (0.2-3.0% per decade, p=0.03), which was also present in the multivariate model (1.6 (0.1-3.2) % per decade, p<0.04). Shunt and alveolar deadspace are shown as a function of time since acute SARS-CoV-2 infection for each NIH severity category in FIG. 6.

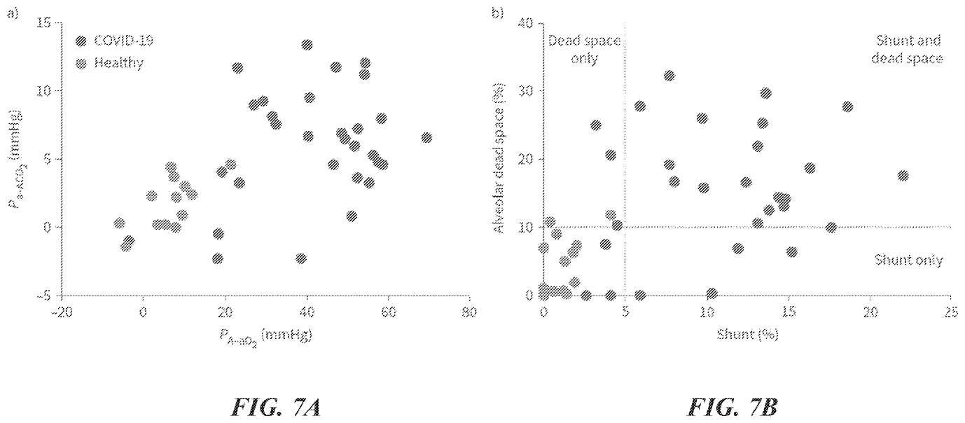

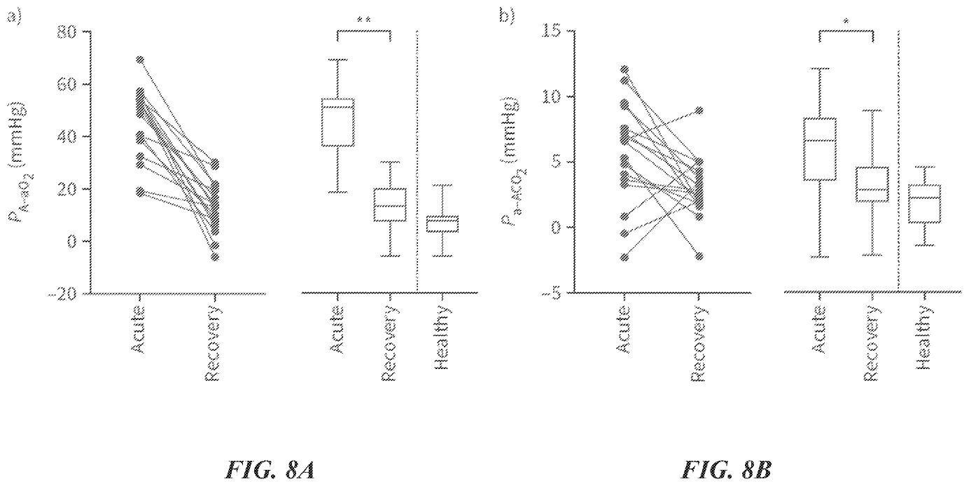

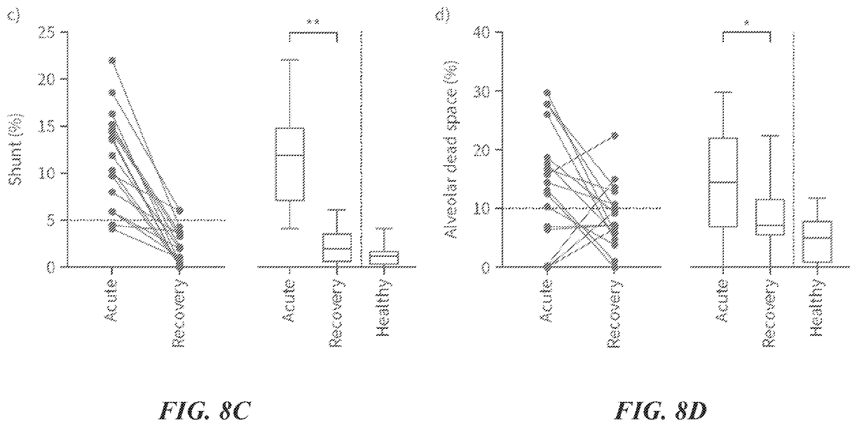

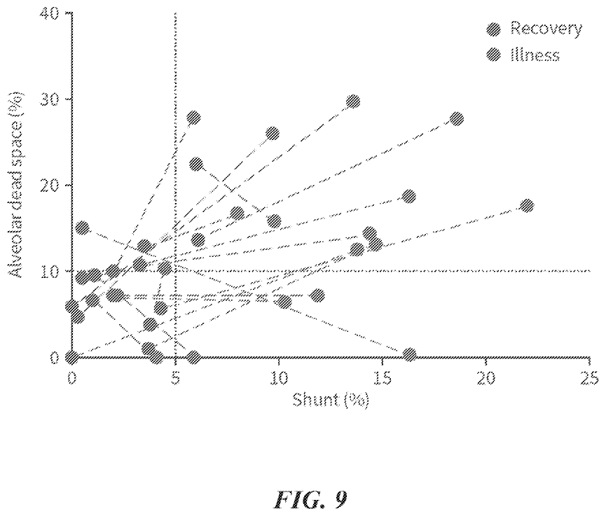

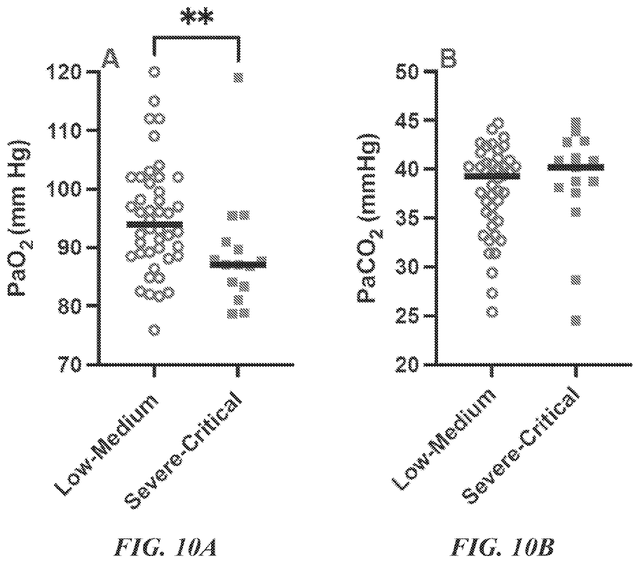

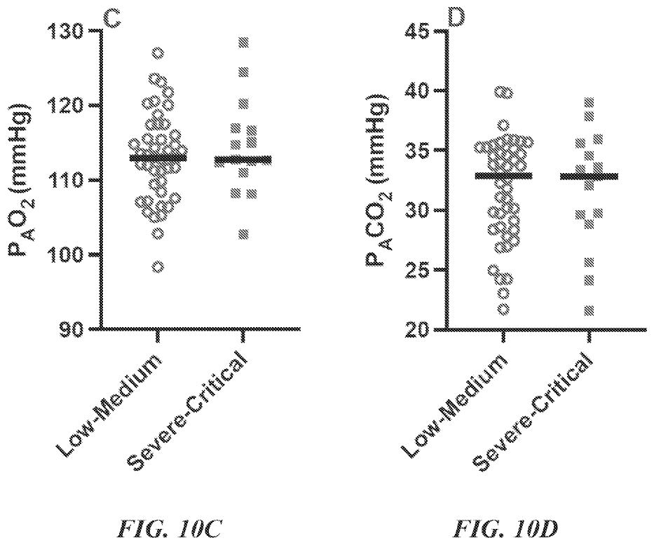

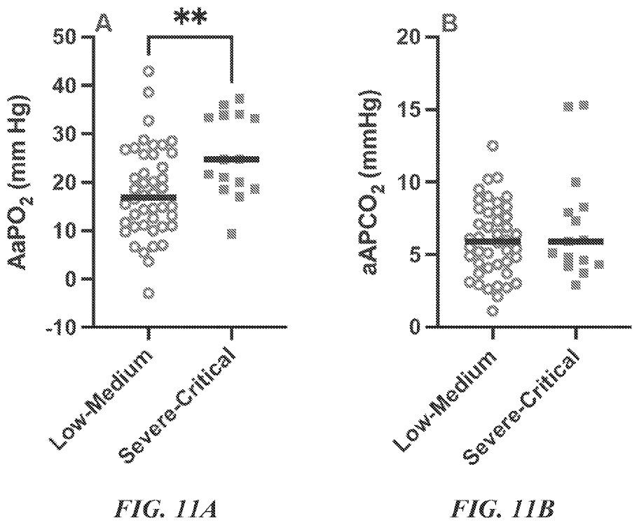

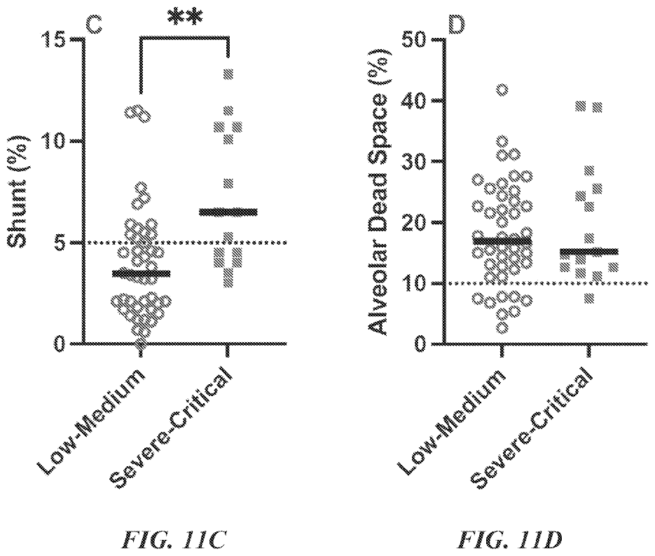

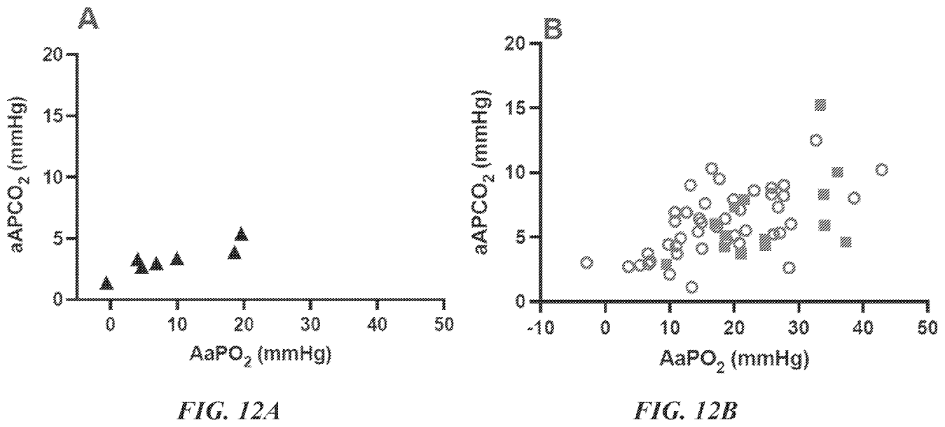

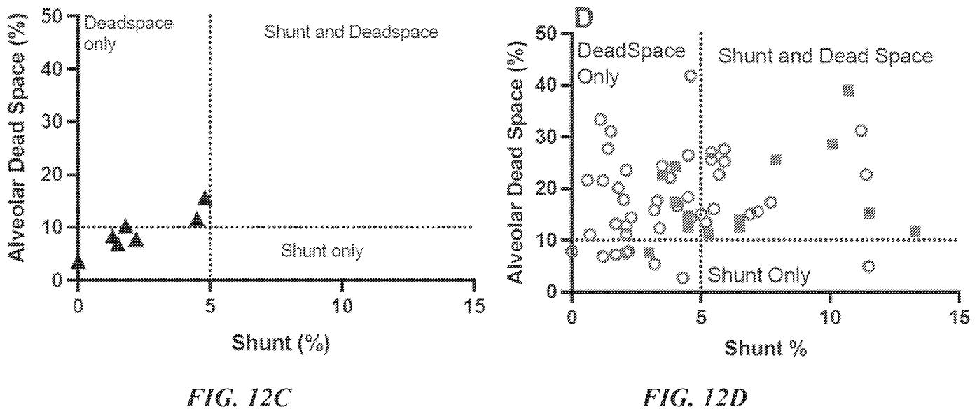

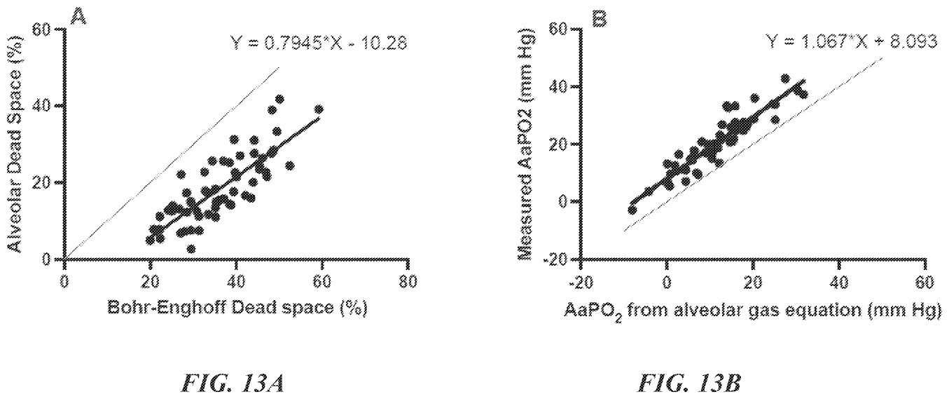

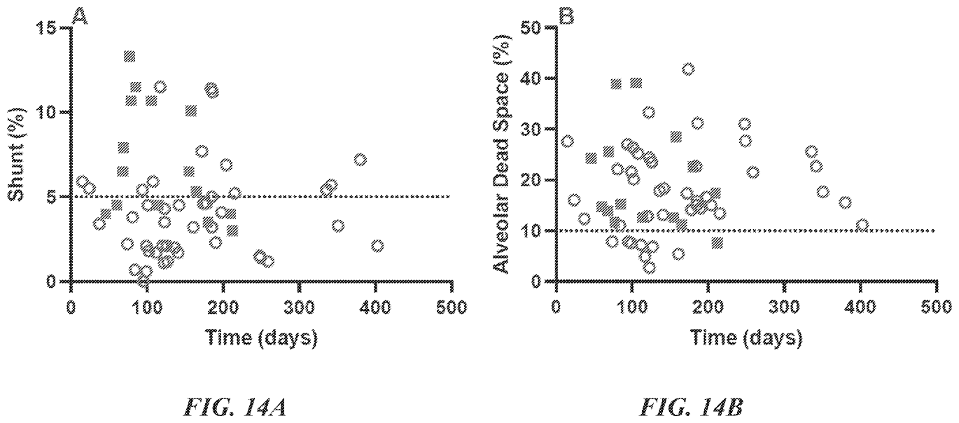

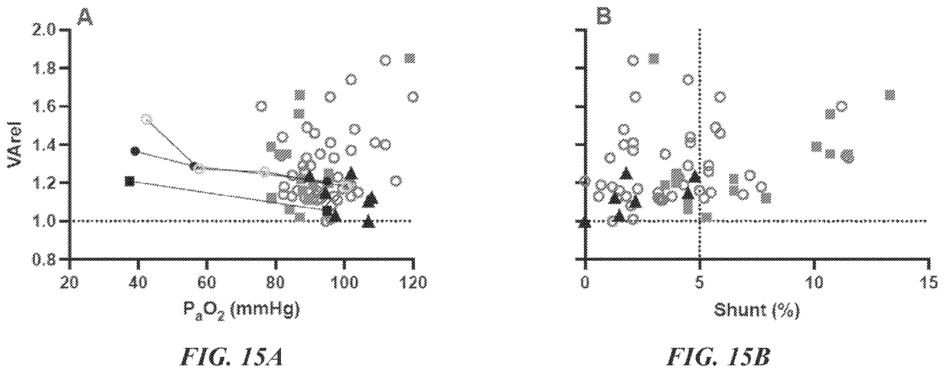

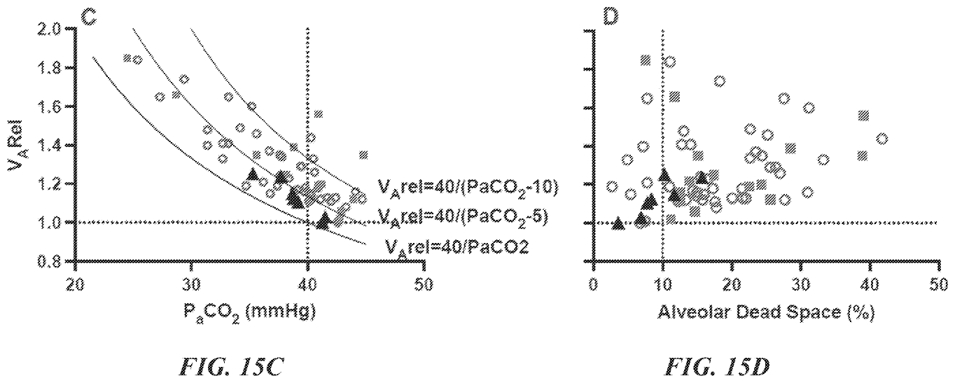

Relative Alveolar Ventilation Ventilation was commonly elevated above expected levels for the post COVID-19 patients (FIG. 7), and this finding was seen at all values of PaO₂, PaCO2, shunt and alveolar deadspace (FIG. 7). There was a significant correlation between VArel and PaCO2 (r−0.76, p<0.0001 and between VArel and shunt (r 0.27, p<0.05).

There were no differences in VArel between patients who complained of breathlessness to those who did not (p=0.1).

Using a recently developed methodology measuring exhaled gas and simultaneous arterial gas measurements to quantify intrapulmonary shunt and alveolar deadspace (65, 72, 76), the principal, novel, findings of our study are: 1) up to 403 days following an acute COVID-19 illness, intrapulmonary shunt remains increased in 37% of patients with alveolar deadspace elevated in 86%; 2) shunt and alveolar deadspace were not correlated, reinforcing the suggestion that they reflect independent pathological processes, 3) elevated shunt was partially related to the severity of the acute illness, while increased alveolar deadspace was not. However, alveolar deadspace was weakly related to increasing age. Based on multivariate analysis, there were no other predictors for shunt or deadspace, including time since acute infection, BMI and comorbid disease. 4) The majority of the patients had increased ventilation regardless of history of disease severity, which was not related to symptoms.

Contrary to our hypothesis, time since acute infection with SARS-CoV-2 (15 days-13 months) was not associated with a reduction in shunt or alveolar deadspace, although there are limited data beyond 200 days. This finding suggests persistence of COVID-19 pulmonary pathology such as pulmonary fibrosis, chronic pulmonary vascular obstruction or angiogenesis (67, 69, 84).

In contrast to our previous study (65), the majority of patients in this study have persistent gas exchange abnormalities at recovery. The patients in this current study were studied later, were more obese, of a similar age and more women were included. This study also included more severe patients, and more patients required intubation and ventilation. These characteristics may be contributing to the greater prevalence of gas exchange abnormalities seen in this current study. In addition, it is likely there is an over-representation of symptomatic patients as these patients remained in contact with health care providers.

Measurement of Intrapulmonary Shunt and Deadspace The technique used in to quantify intra-pulmonary shunt and alveolar deadspace was developed by Wagner et al (72). Methodological controls in this study fell within or close to published values for the 95% confidence levels for shunt (5%), and alveolar deadspace (10%) in healthy adults (73). The advantages of this technique are that it directly measures alveolar gas, allowing accurate estimation of intrapulmonary shunt and alveolar deadspace. It can be used in the setting of an infectious illness, as we have previously shown in acute COVID-19 (65). When compared to estimates of Bohr-Enghoff deadspace, our measurements of alveolar deadspace are less, likely as a consequence of anatomical deadspace included in the Bohr-Enghoff deadspace. Alveolar gas equation estimates of alveolar-arterial oxygen difference also underestimate the true difference. This is a consequence of the alveolar-arterial carbon dioxide difference. In a recent editorial, this methodology was described as a big step forward in gas exchange physiology which should be applied to other pulmonary conditions (76). Indeed, this methodology represents a simple bedside test which can be used more widely than other methods such as multiple inert gas elimination technique (85).

Intrapulmonary Shunt As we hypothesized, patients with more severe disease were more likely to have a larger shunt. The NIH severity category largely uses measures of oxygenation to categorise severity (75), suggesting its construction reflects the magnitude of shunts in the lung. Shunt is a consequence of many pathological processes including: 1) alveolar filling with fluid or cell debris, 2) atelectasis or 3) small airways obstruction that alone or in combination reduce or eliminate ventilation of affected alveoli. From the medical record, most patients had a clear chest radiograph at follow up, while 25% showed some atelectasis. This finding suggests that post COVID-19, alveolar collapse/and or airway filling may not be the cause of increased intra-pulmonary shunt. However, it is worth noting that open but poorly ventilated alveoli could contribute to widening the AaPO₂, and thus to measured shunt, may not be visible on CXR. An increase in shunt may also be a consequence of an increase in anastomotic blood vessels, with angiogenesis and new blood vessel formation reported in the acute illness (84), which if they bypassed alveolar gas would contribute to shunt.

Alveolar Deadspace The increase in alveolar deadspace implies reduced regional alveolar perfusion relative to the ventilation and may be a consequence of persistent vascular damage, redistribution of blood flow from vascular obstruction or hypoxic pulmonary vasoconstriction in those portions of the lung. Micro-embolic disease is reported in the acute infection (84, 86, 87), and plasma samples in post COVID-19 patients with persistent COVID-19 symptoms 6 months after their acute illness have increased micro-clots in their circulation (88). Additionally, increased deadspace may be a consequence of “micro-ischemia”, leading to fibrotic remodelling (89-91). Recently an increase in alveolar deadspace in hospitalised patients recovering from COVID-19 has been reported using computed cardiopulmonography (92). Unlike our study, there was a relationship with severity of disease, with patients treated in ICU more likely to have an increase in alveolar deadspace. Many of the patients included in our study had mild disease, yet despite this had increased alveolar deadspace during recovery, suggesting that this finding may be a common sequel to an acute COVID-19 infection rather than an outcome only of severe disease.

Age was a weak predictor of an increase in alveolar deadspace in post COVID-19 patients. The effect of age may be due to the known age-related increase in alveolar deadspace (93). COVID-19 severity has a known interaction with age and is more likely to be fatal in older people (94, 95). However, the effect of age itself was very small (only a 1.6% increase in alveolar deadspace per decade) in comparison to the degree of elevation of deadspace, and cannot explain the majority of the increased alveolar deadspace. Thus, predictors of the increase in alveolar deadspace, which was highly variable, and as high as 30-40% in some post COVID-19 patients remain unknown.

Alveolar Ventilation Relative ventilation was higher than expected in the majority of patients regardless of disease severity, similar to patients with acute COVID-19 pneumonitis (74), and none had a relative ventilation less than 1. None of the methodological controls were hyperventilating. For post COVID-19 patients, there was no relationship to the degree of hypoxemia, and FIG. 7(A) shows that arterial PaO₂ exceeds 75 mmHg in all patients, and furthermore has no correlation with PaO₂. The frequency of elevated arterial-alveolar CO₂ gradients in post COVID-19 patients is evident from FIG. 7(C), as all the measurements lie above and aACO2 of 0. There was a weak correlation between shunt and relative ventilation, suggesting that increased shunt was partially contributing to the increase in ventilation. There was no relationship with the increase in alveolar deadspace, which was unexpected. Post COVID-19, many patients report an increase in breathlessness (96). In our patient cohort, at least 45% of the patients complained of some degree of breathlessness, there were no differences in deadspace or shunt between patients with shortness of breath and those without, although breathlessness was not quantified systematically. The mechanisms that may explain the increase in ventilation post COVID-19 have been previously discussed in the setting of acute COVID-19 pneumonitis (74). These included genetic differences in hypoxic ventilatory response (HVR) (97, 98), previous sustained hypoxemia resulting in an increase in HVR (99), invasion of the carotid body (97), or central nervous system (100) by SARS-COV-2, or disease-related factors impacting on ventilatory control or respiratory mechanics. As the majority of these patients have no history of significant hypoxemia, chronic changes in the HVR seem unlikely, as does mechanical changes in the lung. Additional studies would be required to establish the roles of these contributing factors. It remains unexplained why such a large proportion of post COVID-19 patients have elevated alveolar ventilation.

The present study defined the 95% confidence upper limit for normal values for intra-pulmonary shunt as <5% and for normal alveolar deadspace as <10%. These limits were based on published values for healthy subjects determined using the multiple inert gas elimination technique (MIGET) (73). The rationale for using these data is as follows. When coupled with West's computer program (79), MIGET input data sets allow prediction of PaO₂, PaCO2, mean alveolar PO₂ and PCO₂ and physiological shunt and alveolar deadspace. Our input data were analysed using the same software (79). This means that the processing of the raw data inputs from both studies is identical. Consequently, the published MIGET data (73) do provide directly appropriate comparator reference values for use in the present study.

Measurement error of aAPCO₂ (a small difference between two large numbers) may have dominated the measurements, contributing to error. If so, it would have been evident as many zero or negative aAPCO₂ values, however, that was not frequently seen. Rather, the values obtained from the methodological controls under the same conditions all fell within, or close to, the 95% confidence levels, indicating the methodology is technically accurate.

Relative ventilation was quantified as 40/alveolar PCO₂. In contrast to more standard methods of quantifying ventilation such as VE/CO₂, this method of quantifying ventilation represents the ventilation of the alveoli alone because alveolar PCO₂ is measured from the expired gas alveolar plateau recorded from each breath. None of the methodological controls had an increase in relative ventilation, again indicating that the methodology is technically accurate.

This is a cross-sectional study, and findings may vary within an individual over time. Patients were studied early in the pandemic, and prior to introduction of vaccination or specific anti-viral medications, which may modify the response/recovery to infection. Patients were recruited from a clinic that was established to follow up all patients who had presented to hospitals in western Sydney. This may have resulted in a selection bias, with greater recruitment of symptomatic patients who may have had an increased prevalence of gas exchange abnormalities than patients without symptoms, possibly resulting in an over-representation of gas exchange abnormalities. The majority of our patients contracted COVID-19 in 2020, and these findings may not be generalisable to more recent viral strains. In addition, there may be an effect of different therapies on the results, although as the majority of our cohort were not hospitalised and did not receive any particular therapy this seems unlikely. In addition, we do not have control data measuring shunt and deadspace with increasing age and BMI, and it may be that these statistical associations are not a consequence of COVID-19.

Conclusions and Clinical Implications Using direct measurement of the alveolar-arterial differences to quantify intrapulmonary shunt and alveolar deadspace (72), we demonstrated 15-403 days after an infection with SARS-CoV-2 alveolar deadspace was elevated in 86% of patients, and increased intrapulmonary shunt in 37% of patients. The time since infection was not a predictor of either increased alveolar deadspace or intrapulmonary shunt, suggesting persistent pulmonary vascular and parenchymal pathology, with shunt likely resulting from alveolar and airway damage or relatively increased perfusion, and deadspace likely resulting from micro-emboli causing loss of alveolar perfusion. These abnormalities may contribute to ongoing COVID-19 symptoms, and may in part also explain why post COVID-19 patients are at risk of readmission for respiratory illness (101).

NIH COVID-19 severity, although predictive of shunt in multivariate models, does not cleanly separate patients with lower shunts from those with higher shunts, and, importantly, does not identify patients with high alveolar deadspace, suggesting that persistent parenchymal and/or pulmonary (micro) vascular pathology occurs regardless of acute COVID-19 severity. An increase in relative alveolar ventilation, associated with higher shunts, was present in the majority of post COVID-19 patients, and may contribute to some of the dyspnea and breathlessness which is reported in between 7-61% of post COVID-19 patients (96, 102-104).

It remains to be determined whether the shunt, alveolar deadspace and ventilation abnormalities observed in some post COVID-19 patients are related to the cluster of symptoms known as “long COVID” (96, 105), and whether they persist even longer or eventually resolve over time.

### Example 7: Ventilation is not Depressed in Patients with Hypoxemia and Acute COVID-19 Infection

Early reports of patients with hypoxemia and coronavirus disease (COVID-19) pneumonia exhibiting little respiratory distress have prompted the suggestion that severe acute respiratory syndrome coronavirus 2 (SARS-CoV-2) infection results in a unique respiratory pathophysiology (106). One hypothesis to explain the apparent disconnect between severe hypoxemia and the reported absence of dyspnea is a blunted hypoxic ventilatory response (HVR).

The hypothesis tested was whether patients with hypoxemia and COVID-19 have reduced ventilation compared with healthy control subjects. As part of a cross-sectional study of gas exchange in patients with early COVID-19 pneumonia on presentation to the hospital, we measured mean alveolar partial pressure of CO₂ (P_{A}_{_{CO2}}), which represents the inverse of alveolar ventilation (\dot{V}A) and related it to the severity of hypoxemia as measured by Pa_{O}_{₂}. Published healthy subject data relating P_{A}_{_{CO2}} to Pa_{O2} under normoxic and acute hypoxic conditions (107-109) were used to assess whether \dot{V}A levels of patients with COVID-19 were in the expected range for the severity of hypoxemia, thus inferring the ventilatory response of these patients.

Thirty spontaneously breathing symptomatic patients admitted to Danderyd Hospital, Stockholm, Sweden, who were >18 years of age, had a positive PCR result for COVID-19, and had Sa_{O2} levels of <96% were included. Patients unable to maintain constant V_{T} and breathing frequency over the data collection period (of several breaths) were excluded. All patients gave written informed consent.

Pa_{O2} and Pa_{CO2} were measured from an arterial blood sample collected over two or three steady-state breaths while the patient was breathing ambient air. Immediately before collecting the blood sample, exhaled CO₂ concentrations and gas flow were measured at 100 Hz at the mouth (Oxycon Pro; Vyaire Medical (110)), and, after adjustment for analyzer lag, mean P_{A}_{_{CO2}} was determined as the average of three separate breaths. Measured Pa_{O2} and Pa_{CO2} were corrected to body temperature (111), and exhaled gas measurements were also temperature adjusted using the Antoine equation.

The term 40/P_{A}_{_{CO2}} was calculated (indicating the \dot{V}A relative to that which would be present in the same patient had P_{A}_{_{CO2}} been normal at 40 mm Hg). This term, abbreviated to \dot{V}Arel, was plotted against Pa_{O2}.

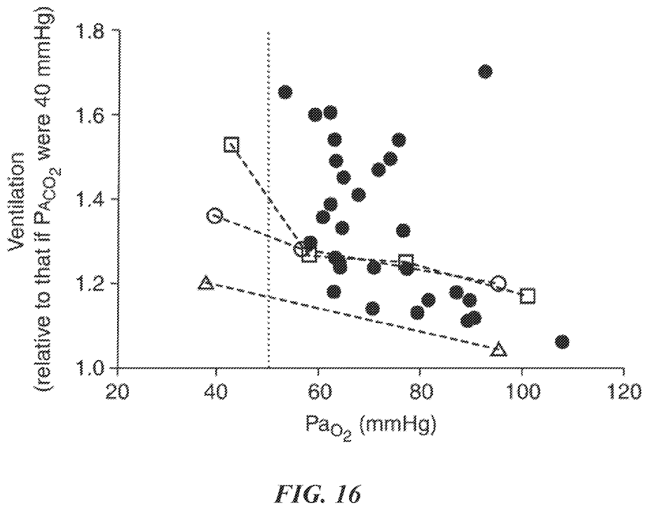

Data were collected from 22 males and 8 females, aged 23-85 years (mean±SD, 50.7±15.0 yr). All subjects had mildly symptomatic COVID-19 pneumonia; the majority were tachypneic (respiratory rate, 21.8±7.2 breaths/min; range, 9-38), 22 had dyspnea, and most were febrile at the time of testing (body temperature, 38.0±1.0° C.; range, 36.5-40° C.). No patient required ICU admission. Exhaled CO₂ was collected between 2 and 100 seconds (mean±SD, 35±10 s) before the arterial blood gas sample. Pa_{O2} ranged from 52.9 to 107.5 mm Hg (mean±SD, 72.0±12.7 mm Hg), arterial oxygen saturation ranged from 89% to 99% (mean±SD, 94±2%), Pa_{CO2} ranged from 27.8 to 46.8 mm Hg (mean±SD, 36.3±4.6 mm Hg), and \dot{V}Arel ranged from 1.1 to 1.7 (mean±SD, 1.3±0.2). Approximately 50% of patients had \dot{V}Arel values that were in broad agreement with normal values (FIG. 16). For all remaining patients, \dot{V}Arel was greater than expected from the normal data. Most importantly, in no patient was \dot{V}Arel lower than that seen in healthy subjects at any Pa_{O2}.

The findings demonstrate that in this group of 30 patients with acute symptomatic COVID-19, \dot{V}A was normal or increased at any Pa_{O2} as compared with that in healthy subjects exposed to acute hypoxia. Contrary to our hypothesis, no patient had evidence of reduced or blunted ventilation. Notably, all patients had Pa_{O2}>50 mm Hg, the nominal level below which hypoxia-driven dyspnea and ventilation increase rapidly in healthy subjects (FIG. 16) (112). To our knowledge, these findings represent the first report with data showing that ventilatory responsiveness in spontaneously ambient air-breathing patients with COVID-19 is normal or increased and not decreased. A strength of our study is that our patient data are based on a direct, noninvasive measurement of P_{A}_{_{CO2}} from exhaled gas analysis, thus providing a surrogate measurement of \dot{V}_{A} that is obtainable at the bedside in a clinical infectious disease setting.

This is an observational cross-sectional study, and we are unable to determine the mechanism contributing to the observed V′_{A}rel data. Potential mechanisms influencing ventilatory drive in COVID-19 pneumonia include the following: 1) genetically determined differences in HVR (113, 114); 2) sustained hypoxemia over a period of hours to days, resulting in an increase in HVR (via the hypoxia-inducible factor hydroxylase system) (115); 3) SARS-CoV-2 invasion of the carotid body or central nervous system, resulting in direct changes in ventilatory response (113); and 4) other disease-related but not COVID-19-specific factors affecting ventilatory control (e.g., sensory receptor inputs, fever, anxiety, pain, inflammation) or respiratory mechanics. Additional studies would be required to establish the roles of these contributing factors.

We recruited spontaneously breathing, symptomatic, hospitalized patients with COVID-19 who were judged by their caregivers to be safe while breathing ambient air for the few minutes of the study, and our findings may not be generalizable to other COVID-19 disease stages or severities. We did not perform classical HVR protocols, with control of inhaled gases and direct measurement of ventilation in each individual, because of logistical challenges in a highly infectious acute disease setting. Our data consist of a single sample for each patient, and we do not know where each subject is operating in their intrinsic HVR relationship. We also have not included concurrent healthy or non-COVID-19 pneumonia control subjects and have used historical control subjects from three prior physiological studies in which healthy young subjects were studied over the same range of Pa_{O2} levels as encountered in our patients with COVID-19.

Patients with acute COVID-19 (spontaneously breathing ambient air) do not have depressed \dot{V}_{A} relative to their degree of hypoxemia. Indeed, some patients have relatively high ventilatory levels. These findings do not support the concept of impaired HVR in COVID-19 pneumonia.

### REFERENCES

1. Rosas 10, Brau N, Waters M, Go R C, Hunter B D, Bhagani S, Skiest D, Aziz M S, Cooper N, Douglas I S, Savic S, Youngstein T D, Sorbo L, C, Gracian A, De La Zerda D J, Ustianowski A, Bao M, Dimonaco S, GrahamE, Matharu B, Spotswood H, Tsai L, Malhotra A. Tocilizumab in hospitalized patients with severe COVID-19 pneumonia. N Engl J Med 384: 1503-1516, 2021. doi:10.1056/NEJMoa2028700.
2. Wagner P D, Laravuso R B, Uhl R R, West J B. Distributions of ventilation-perfusion ratios in acute respiratory failure. Chest 65: 32S-35S, 1974. doi:10.1378/chest.65.4_Supplement.32S.
3. Ackermann M, Verleden S E, Kuehnel M, Haverich A, Welte T, Laenger F, Vanstapel A, Werlein C, Stark H, Tzankov A, Li W W, Li V W, Mentzer S J, Jonigk D. Pulmonary vascular endothelialitis, thrombosis, and angiogenesis in Covid-19. N Engl J Med 383: 120-128, 2020. doi:10.1056/NEJMoa2015432.
4. D′Alonzo G E, Bower J S, DeHart P, Dantzker D R. The mechanisms of abnormal gas exchange in acute massive pulmonary embolism. Am Rev Respir Dis 128: 170-172, 1983. doi:10.1164/arrd.1983.128.1.170.
5. Rahn H, Fenn W. A Graphical Analysis of the Respiratory Gas Exchange. Washington, D C: American Physiological Society, 1955.
6. Simonson T S, Baker T L, Banzett R B, Bishop T, Dempsey J A, Feldman J L, Guyenet P G, Hodson E J, Mitchell G S, Moya E A, Nokes B T, Orr J E, Owens R L, Poulin M, Rawling J M, Schmickl C N, Watters J J, Younes M, Malhotra A. Silent hypoxaemia in COVID-19 patients. J Physiol 599: 1057-1065, 2021. doi:10.1113/JP280769.
7. Norderfeldt J, Liliequist A, Frostell C, Adding C, Agvald P, Eriksson M, Lonnqvist P A. Acute pulmonary hypertension and short-term outcomes in severe Covid-19 patients needing intensive care. ActaAnaesthesiol Scand 65: 761-769, 2021. doi:10.1111/aas.13819.
8. Zhang Y, Sun W, Wu C, Zhang Y, Cui L, Xie Y, Wang B, He L, Yuan H, Zhang Y, Cai Y, Li M, Zhang Y, Yang Y, Li Y, Wang J, Yang Y, Lv Q, Zhang L, Xie M. Prognostic value of right ventricular ejection fraction assessed by 3D echocardiography in COVID-19 patients. Front Cardiovasc Med 8: 641088, 2021. doi:10.3389/fcvm.2021.641088.
9. Mishra A, Lal A, Sahu K K, George A A, Martin K, Sargent J. An update on pulmonary hypertension in coronavirus disease-19 (COVID-19). Acta Biomed 91: e2020155, 2020. doi:10.23750/abm.v91 i4.10698.
10. Kubo K, Ge R L, Koizumi T, Fujimoto K, Yamanda T, Haniuda M, Honda T. Pulmonary artery remodeling modifies pulmonary hypertension during exercise in severe emphysema. Respir Physiol 120:71-79, 2000. doi:10.1016/s0034-5687(00)00090-6.
11. Evans J W, Wagner P D, West J B. Conditions for reduction of pulmonary gas transfer by ventilation-perfusion inequality. J Appl Physiol 36: 533-537, 1974. doi:10.1152/jappl.1974.36.5.533.
12. West J B, Wagner P D, Derks C M. Gas exchange in distributions of \dot{V}A-Q ratios: partial pressure-solubility diagram. J Appl Physiol 37:533-540, 1974. doi:10.1152/jappl.1974.37.4.533.
13. Hopkins S R, Wagner P D. The Multiple Inert Gas Elimination Technique. New York: Springer, 2017.
14. Wagner P D, Saltzman H A, West J B. Measurement of continuous distributions of ventilation-perfusion ratios: theory. J Appl Physiol 36: 588-599, 1974. doi:10.1152/jappl.1974.36.5.588.
15. Farhi L E. Elimination of inert gas by the lung. Respir Physiol 3: 1-11, 1967. doi:10.1016/0034-5687(67)90018-7.
16. Riley R L, Cournand A. Ideal alveolar air and the analysis of ventilation-perfusion relationships in the lungs. J Appl Physiol 1: 825-847, 1949. doi:10.1152/jappl.1949.1.12.825.
17. West J B. Ventilation-perfusion inequality and overall gas exchange in computer models of the lung. Respir Physiol 7: 88-110, 1969. doi:10.1016/0034-5687(69)90071-1.
18. Kelman G R. Digital computer subroutine for the conversion of oxygen tension into saturation. J Appl Physiol 21: 1375-1376, 1966. doi:10.1152/jappl.1966.21.4.1375.
19. Kelman G R. Digital computer procedure for the conversion of PCO₂ into blood CO₂ content. Respir Physiol 3: 111-115, 1967. doi:10.1016/0034-5687(67)90028-x.
20. Kelman G R, Nunn J F. Nomograms for correction of blood Po2, Pco2, pH, and base excess for time and temperature. J Appl Physiol 21: 1484-1490, 1966. doi:10.1152/jappl.1966.21.5.1484.
21. Karbing D S, Panigada M, Bottino N, Spinelli E, Protti A, Rees S E, Gattinoni L. Changes in shunt, ventilation/perfusion mismatch, and lung aeration with PEEP in patients with ARDS: a prospective singlearm interventional study. Crit Care 24: 111, 2020. doi:10.1186/s13054-020-2834-6.
22. Karbing D S, Kjorgaard S, Smith B W, Espersen K, Allerod C, Andreassen S, Rees S E. Variation in the PaO₂/FiO₂ ratio with FiO₂: mathematical and experimental description, and clinical relevance. Crit Care 11: R118, 2007. doi:10.1186/cc6174.
23. Karbing D S, Kjorgaard S, Andreassen S, Espersen K, Rees S E. Minimal model quantification of pulmonary gas exchange in intensive care patients. Med Eng Phys 33: 240-248, 2011. doi:10.1016/j.medengphy.2010.10.007.
24. Boning D, Enciso G. Hemoglobin-oxygen affinity in anemia. Blut 54:361-368, 1987. doi:10.1007/BF00626019.
25. Myburgh J A, Webb R K, Worthley L I. The P50 is reduced in critically ill patients. Intensive Care Med 17: 355-358, 1991. doi:10.1007/BF01716196.
26. Myburgh J A, Webb R K, Worthley L I. Ventilation/perfusion indices do not correlate with the difference between oxygen consumption measured by the Fick principle and metabolic monitoring systems in critically ill patients. Crit Care Med 20: 479-482, 1992. doi:10.1097/00003246-199204000-00008.
27. Astrand P O, Cuddy T E, Saltin B, Stenberg J. Cardiac output during submaximal and maximal work. J Appl Physiol 19: 268-274, 1964. doi:10.1152/jappl.1964.19.2.268.
28. Rowell L B. Human Cardiovascular Control. Oxford: Oxford University Press, 1993.
29. Dubois A B. Alveolar CO2 and O₂ during breath holding, expiration, and inspiration. J Appl Physiol 5: 1-12, 1952. doi:10.1152/jappl.1952.5.1.1.
30. Dubois A B, Britt A G, Fenn W O. Alveolar CO₂ during the respiratory cycle. J Appl Physiol 4: 535-548, 1952. doi:10.1152/jappl.1952.4.7.535.
31. Dubois A B, Fenn W O, Britt A G. CO₂ dissociation curve of lung tissue. J Appl Physiol 5: 13-16, 1952. doi:10.1152/jappl.1952.5.1.13.
32. Dubois A B, Fowler R C, Soffer A, Fenn W O. Alveolar CO₂ measured by expiration into the rapid infrared gas analyzer. J Appl Physiol 4: 526-534, 1952. doi:10.1152/jappl.1952.4.7.526.
33. Fowler W S. Lung function studies; the respiratory deadspace. Am J Physiol 154: 405-416, 1948. doi:10.1152/ajplegacy.1948.154.3.405.
34. Fowler W S. Respiratory deadspace. Fed Proc 7: 35, 1948.
35. Wagner P D, Hedenstierna G, Bylin G. Ventilation-perfusion inequality in chronic asthma. Am Rev Respir Dis 136: 605-612, 1987.doi:10.1164/ajrccm/136.3.605.
36. Chew M S, Blixt P J, Ahman R, et al. National outcomes and characteristics of patients admitted to Swedish intensive care units for COVID-19: a registry-based cohort study. Eur J Anaesthesiol 2021; 38: 335-343.
37. Gattinoni L, Coppola S, Cressoni M, et al. COVID-19 does not lead to a “typical” acute respiratory distress syndrome. Am J Respir Crit Care Med 2020; 201: 1299-1300.
38. Ottestad W, Sovik S. COVID-19 patients with respiratory failure: what can we learn from aviation medicine?Br J Anaesth 2020; 125: e280-e281.
39. Meng H, Xiong R, He R, et al. C T imaging and clinical course of asymptomatic cases with COVID-19 pneumonia at admission in Wuhan, China. J Infect 2020; 81: e33-e39.
40. Gattinoni L, Chiumello D, Caironi P, et al. COVID-19 pneumonia: different respiratory treatments for different phenotypes?Intensive Care Med 2020; 46: 1099-1102.
41. Kairaitis K, Harbut P, Hedenstierna G, et al. Ventilation is not depressed in patients with hypoxemia and acute COVID-19 infection. Am J Respir Crit Care Med 2022; 205: 1119-1120.
42. Ackermann M, Verleden S E, Kuehnel M, et al. Pulmonary vascular endothelialitis, thrombosis, and angiogenesis in Covid-19. N Engl J Med 2020; 383: 120-128.
43. Leisman D E, Deutschman C S, Legrand M. Facing COVID-19 in the ICU: vascular dysfunction, thrombosis, and dysregulated inflammation. Intensive Care Med 2020; 46: 1105-1108.
44. Magro C, Mulvey J J, Berlin D, et al. Complement associated microvascular injury and thrombosis in the pathogenesis of severe COVID-19 infection: a report of five cases. Transl Res 2020; 220: 1-13.
45. Wagner P D, Malhotra A, Prisk G K. Using pulmonary gas exchange to estimate shunt and deadspace in lung disease: theoretical approach and practical basis. J Appl Physiol 2022; 132:1104-1113.
46. Bradley A F, Severinghaus J W, Stupfel M. Effect of temperature on PCO₂ and PO2 of blood in vitro. J Appl Physiol 1956; 9: 201-204.
47. Antoine C. Tension des vapeurs: nouvelle relation entre les tension et les temperatures. Comptes Rendus 1888; 107: 681-684, 778-780, 836-837.
48. Riley R L, Cournand A. ‘Ideal’ alveolar air and the analysis of ventilation-perfusion relationships in the lungs. J Appl Physiol 1949; 1: 825-847.
49. Astrand P O, Cuddy T E, Saltin B, et al. Cardiac output during submaximal and maximal work. J Appl Physiol 1964; 19: 268-274.
50. Rowell L B. Human Cardiovascular Control. New York, Oxford University Press, 1993.
51. West J B. Ventilation-perfusion inequality and overall gas exchange in computer models of the lung. Respir Physiol 1969; 7: 88-110.
52. Wagner P D, Hedenstierna G, Bylin G. Ventilation-perfusion inequality in chronic asthma. Am Rev Respir Dis 1987; 136: 605-612.
53. Dantzker D R, Brook C J, Dehart P, et al. Ventilation-perfusion distributions in the adult respiratory distress syndrome. Am Rev Respir Dis 1979; 120: 1039-1052.
54. Herrmann J, Mori V, Bates J H T, et al. Modeling lung perfusion abnormalities to explain early COVID-19 hypoxemia. Nat Commun 2020; 11: 4883.
55. Tian S, Hu W, Niu L, et al. Pulmonary pathology of early-phase 2019 novel coronavirus (COVID-19) pneumonia in two patients with lung cancer. J Thorac Oncol 2020; 15: 700-704.
56. Kianzad A, Meijboom L J, Nossent E J, et al. COVID-19: histopathological correlates of imaging patterns on chest computed tomography. Respirology 2021; 26: 869-877.
57. Price L C, Ridge C, Wells A U. Pulmonary vascular involvement in COVID-19 pneumonitis: is this the first and final insult?Respirology 2021; 26: 832-834.
58. Katsoularis I, Fonseca-Rodriguez O, Farrington P, et al. Risks of deep vein thrombosis, pulmonary embolism, and bleeding after Covid-19: nationwide self-controlled cases series and matched cohort study. BMJ 2022; 377: e069590.
59. The ATTACC, ACTIV-4a and REMAP-CAP Investigators. Therapeutic anticoagulation with heparin in noncritically ill patients with Covid-19. N Engl J Med 2021; 385: 790-802.
60. The REMAP-CAP, ACTIV-4a and ATTACC Investigators. Therapeutic anticoagulation with heparin in critically ill patients with Covid-19. N Engl J Med 2021: 385: 777-789.
61. Radermacher P, Herigault R, Teisseire B, et al. Low V A/Q areas: arterial-alveolar N2 difference and multiple inert gas elimination technique. J Appl Physiol 1988; 64: 2224-2229.
62. Hopkins S R. Functional magnetic resonance imaging of the lung: a physiological perspective. J Thorac Imaging 2004; 19: 228-234.
63. Zhu N, Zhang D, Wang W, Li X, Yang B, Song J, Zhao X, Huang B, Shi W, Lu R, Niu P, Zhan F, Ma X, Wang D, Xu W, Wu G, Gao G F, Tan W, China Novel Coronavirus I, and Research T. A Novel Coronavirus from Patients with Pneumonia in China, 2019. N Engl J Med 382:727-733, 2020.
64. Habashi N M, Camporota L, Gatto L A, and Nieman G. Functional pathophysiology of SARS-CoV-2-induced acute lung injury and clinical implications. J Appl Physiol (1985) 130: 877-891, 2021.
65. Harbut P, Prisk G K, Lindwall R, Hamzei S, Palmgren J, Farrow C E, Hedenstierna G, Amis T C, Malhotra A, Wagner P D, and Kairaitis K. Intrapulmonary shunt and alveolar deadspace in a cohort of patients with acute COVID-19 pneumonitis and early recovery. Eur Respir J 61: 2023.
66. Huang C, Wang Y, Li X, Ren L, Zhao J, Hu Y, Zhang L, Fan G, Xu J, Gu X, Cheng Z, Yu T, Xia J, Wei Y, Wu W, Xie X, Yin W, Li H, Liu M, Xiao Y, Gao H, Guo L, Xie J, Wang G, Jiang R, Gao Z, Jin Q, Wang J, and Cao B. Clinical features of patients infected with 2019 novel coronavirus in Wuhan, China. Lancet 395: 497-506, 2020.
67. Tarraso J, Safont B, Carbonell-Asins J A, Fernandez-Fabrellas E, Sancho-Chust J N, Naval E, Amat B, Herrera S, Ros J A, Soler-Cataluna J J, Rodriguez-Portal J A, Andreu A L, Marin M, Rodriguez-Hermosa J L, Gonzalez-Villaescusa C, Soriano J B, Signes-Costa J, and team C-Fs. Lung function and radiological findings 1 year after COVID-19: a prospective follow-up. Respir Res 23: 242, 2022.
68. Taverne J, Salvator H, Leboulch C, Barizien N, Ballester M, Imhaus E, Chabi-Charvillat M L, Boulin A, Goyard C, Chabrol A, Catherinot E, Givel C, Couderc L J, and Tcherakian C. High incidence of hyperventilation syndrome after COVID-19. J Thorac Dis 13:3918-3922, 2021.
69. Hama Amin B J, Kakamad F H, Ahmed G S, Ahmed S F, Abdulla B A, Mohammed S H, Mikael T M, Salih R O, Ali R K, Salh A M, and Hussein D A. Post COVID-19 pulmonary fibrosis; a meta-analysis study. Ann Med Surg (Lond) 77: 103590, 2022.
70. Grist J T, Chen M, Collier G J, Raman B, Abueid G, McIntyre A, Matthews V, Fraser E, Ho L P, Wild J M, and Gleeson F. Hyperpolarized (129)Xe MRI Abnormalities in Dyspneic Patients 3 Months after COVID-19 Pneumonia: Preliminary Results. Radiology 301: E353-E360, 2021.
71. Grist J T, Collier G J, Walters H, Kim M, Chen M, Abu Eid G, Laws A, Matthews V, Jacob K, Cross S, Eves A, Durrant M, McIntyre A, Thompson R, Schulte R F, Raman B, Robbins P A, Wild J M, Fraser E, and Gleeson F. Lung Abnormalities Detected with Hyperpolarized (129)Xe MRI in Patients with Long COVID. Radiology 305: 709-717, 2022.
72. Wagner P D, Malhotra A, and Prisk G K. Using pulmonary gas exchange to estimate shunt and deadspace in lung disease: theoretical approach and practical basis. J Appl Physiol (1985) 132: 1104-1113, 2022.
73. Wagner P D, Hedenstierna G, and Bylin G. Ventilation-perfusion inequality in chronic asthma. Am Rev Respir Dis 136: 605-612, 1987.
74. Kairaitis K, Harbut P, Hedenstierna G, Prisk G K, Farrow C E, Amis T, Wagner P D, and Malhotra A. Ventilation Is Not Depressed in Patients with Hypoxemia and Acute COVID-19 Infection. Am J Respir Crit Care Med 205: 1119-1120, 2022.
75. COVID-19 Treatment Guidelines Panel. Coronavirus Disease 2019 (COVID-19) Treatment Guidelines. National Institutes of Health. Available at https://www.covid19treatmentguidelines.nih.gov/. Accessed September 2022.
76. Hughes M. Novel gas exchange analysis in COVID-19 lung disease. Eur Respir J 61: 2023.
77. Bates J H, Prisk G K, Tanner T E, and McKinnon A E. Characterizing and correcting for the dynamic response of a bag-in-box system. J Appl Physiol Respir Environ Exerc Physiol 56: 254-258, 1984.
78. Riley R L, and Cournand A. Ideal alveolar air and the analysis of ventilation-perfusion relationships in the lungs. J Appl Physiol 1: 825-847, 1949.
79. West J B. Ventilation-perfusion inequality and overall gas exchange in computer models of the lung. Respir Physiol 7: 88-110, 1969.
80. Astrand P O, Cuddy T E, Saltin B, and Stenberg J. Cardiac Output during Submaximal and Maximal Work. J Appl Physiol 19: 268-274, 1964.
81. Wagner P D, Gale G E, Moon R E, Torre-Bueno J R, Stolp B W, and Saltzman H A. Pulmonary gas exchange in humans exercising at sea level and simulated altitude. J Appl Physiol (1985) 61: 260-270, 1986.
82. Hammond M D, Gale G E, Kapitan K S, Ries A, and Wagner P D. Pulmonary gas exchange in humans during normobaric hypoxic exercise. J Appl Physiol (1985) 61: 1749-1757, 1986.
83. Torre-Bueno J R, Wagner P D, Saltzman H A, Gale G E, and Moon R E. Diffusion limitation in normal humans during exercise at sea level and simulated altitude. J Appl Physiol (1985) 58: 989-995, 1985.
84. Ackermann M, Verleden S E, Kuehnel M, Haverich A, Welte T, Laenger F, Vanstapel A, Werlein C, Stark H, Tzankov A, Li W W, Li V W, Mentzer S J, and Jonigk D. Pulmonary Vascular Endothelialitis, Thrombosis, and Angiogenesis in Covid-19. N Engl J Med 383: 120-128, 2020.
85. Wagner P D, Saltzman H A, and West J B. Measurement of continuous distributions of ventilation-perfusion ratios: theory. J Appl Physiol 36: 588-599, 1974.
86. Vrints C J M, Krychtiuk K A, Van Craenenbroeck E M, Segers V F, Price S, and Heidbuchel H. Endothelialitis plays a central role in the pathophysiology of severe COVID-19 and its cardiovascular complications. Acta Cardiol 76: 109-124, 2021.
87. Price L C, Ridge C, and Wells A U. Pulmonary vascular involvement in COVID-19 pneumonitis: Is this the first and final insult?Respirology 26: 832-834, 2021.
88. Pretorius E, VIok M, Venter C, Bezuidenhout J A, Laubscher G J, Steenkamp J, and Kell D B. Persistent clotting protein pathology in Long COVID/Post-Acute Sequelae of COVID-19 (PASC) is accompanied by increased levels of antiplasmin. Cardiovasc Diabetol 20:172, 2021.
89. Ackermann M, Mentzer S J, Kolb M, and Jonigk D. Inflammation and intussusceptive angiogenesis in COVID-19: everything in and out of flow. Eur Respir J 56: 2020.
90. Ackermann M, Tafforeau P, Brunet J, Kamp J C, Werlein C, Kuhnel M P, Jacob J, Walsh C L, Lee P D, Welte T, and Jonigk D D. Reply to: Intrapulmonary Shunt and Alveolar Deadspace in a Cohort of Patients with Acute COVID-19 Pneumonitis and Early Recovery. Eur Respir J 2023.
91. Ackermann M, Kamp J C, Werlein C, Walsh C L, Stark H, Prade V, Surabattula R, Wagner W L, Disney C, Bodey A J, Illig T, Leeming D J, Karsdal M A, Tzankov A, Boor P, Kuhnel M P, Langer F P, Verleden S E, Kvasnicka H M, Kreipe H H, Haverich A, Black S M, Walch A, Tafforeau P, Lee P D, Hoeper M M, Welte T, Seeliger B, David S, Schuppan D, Mentzer S J, and Jonigk D D. The fatal trajectory of pulmonary COVID-19 is driven by lobular ischemia and fibrotic remodelling. EBioMedicine 85: 104296, 2022.
92. Magor-Elliott S R M, Alamoudi A, Chamley R R, Xu H, Wellalagodage T, McDonald R P, O'Brien D, Collins J, Coombs B, Winchester J, Sellon E, Xie C, Sandhu D, Fullerton C J, Couper J H, Smith N M J, Richmond G, Cassar M P, Raman B, Talbot N P, Bennett A N, Nicol E D, Ritchie G A D, Petousi N, Holdsworth D A, and Robbins P A. Altered lung physiology in two cohorts after COVID-19 infection as assessed by computed cardiopulmonography. J Appl Physiol (1985) 133: 1175-1191, 2022.
93. Miller R M, and Tenney S M. Deadspace ventilation in old age. J Appl Physiol 9: 321-327, 1956.
94. Romero Starke K, Reissig D, Petereit-Haack G, Schmauder S, Nienhaus A, and Seidler A. The isolated effect of age on the risk of COVID-19 severe outcomes: a systematic review with meta-analysis. BMJ Global Health 6: e006434, 2021.
95. Team C-F. Variation in the COVID-19 infection-fatality ratio by age, time, and geography during the pre-vaccine era: a systematic analysis. Lancet 399: 1469-1488, 2022.
96. Davis H E, Assaf G S, McCorkell L, Wei H, Low R J, Re'em Y, Redfield S, Austin J P, and Akrami A. Characterizing long COVID in an international cohort: 7 months of symptoms and their impact. EClinicalMedicine 38: 101019, 2021.
97. Swenson K E, Ruoss S J, and Swenson E R. The Pathophysiology and Dangers of Silent Hypoxemia in COVID-19 Lung Injury. Ann Am Thorac Soc 18: 1098-1105, 2021.
98. Weil J V. Variation in human ventilatory control-genetic influence on the hypoxic ventilatory response. Respir Physiol Neurobiol 135: 239-246, 2003.
99. Hodson E J, Nicholls L G, Turner P J, Llyr R, Fielding J W, Douglas G, Ratnayaka I, Robbins P A, Pugh C W, Buckler K J, Ratcliffe P J, and Bishop T. Regulation of ventilatory sensitivity and carotid body proliferation in hypoxia by the PHD2/HIF-2 pathway. J Physiol 594:1179-1195, 2016.
100. Monje M, and Iwasaki A. The neurobiology of long COVID. Neuron 110: 3484-3496, 2022.
101. Ayoubkhani D, Khunti K, Nafilyan V, Maddox T, Humberstone B, Diamond I, and Banerjee A. Post-covid syndrome in individuals admitted to hospital with covid-19: retrospective cohort study. BMJ 372: n693, 2021.
102. Sonnweber T, Sahanic S, Pizzini A, Luger A, Schwabl C, Sonnweber B, Kurz K, Koppelstatter S, Haschka D, Petzer V, Boehm A, Aichner M, Tymoszuk P, Lener D, Theurl M, Lorsbach-Köhler A, Tancevski A, Schapfl A, Schaber M, Hilbe R, Nairz M, Puchner B, Huttenberger D, Tschurtschenthaler C, Afßhoff M, Peer A, Hartig F, Bellmann R, Joannidis M, Gollmann-Tepeköylü C, Holfeld J, Feuchtner G, Egger A, Hoermann G, Schroll A, Fritsche G, Wildner S, Bellmann-Weiler R, Kirchmair R, Helbok R, Prosch H, Rieder D, Trajanoski Z, Kronenberg F, W611 E, Weiss G, Widmann G, Löffler-Ragg J, and Tancevski 1. Cardiopulmonary recovery after COVID-19: an observational prospective multicentre trial. Eur Respir J 57: 2021.
103. Tenforde M W, Kim S S, Lindsell C J, Billig Rose E, Shapiro N I, Files D C, Gibbs K W, Erickson H L, Steingrub J S, Smithline H A, Gong M N, Aboodi M S, Exline M C, Henning D J, Wilson J G, Khan A, Qadir N, Brown S M, Peltan I D, Rice T W, Hager D N, Ginde A A, Stubblefield W B, Patel M M, Self W H, and Feldstein L R. Symptom Duration and Risk Factors for Delayed Return to Usual Health Among Outpatients with COVID-19 in a Multistate Health Care Systems Network—United States, March-June 2020. MMWR Morb Mortal Wkly Rep 69: 993-998, 2020.
104. Carfi A, Bernabei R, Landi F, and Group ftGAC-P-ACS. Persistent Symptoms in Patients After Acute COVID-19. JAMA 324: 603-605, 2020.
105. Zhang H, Zang C, Xu Z, Zhang Y, Xu J, Bian J, Morozyuk D, Khullar D, Zhang Y, Nordvig A S, Schenck E J, Shenkman E A, Rothman R L, Block J P, Lyman K, Weiner M G, Carton T W, Wang F, and Kaushal R. Data-driven identification of post-acute SARS-CoV-2 infection subphenotypes. Nat Med 2022.
106. Tobin M J, Laghi F, Jubran A. Why COVID-19 silent hypoxemia is baffling to physicians. Am J Respir Crit Care Med. 2020; 202:356-360.
107. Wagner P D, Gale G E, Moon R E, Torre-Bueno J R, Stolp B W, Saltzman H A. Pulmonary gas exchange in humans exercising at sea level and simulated altitude. J Appl Physiol (1985). 1986; 61:260-270.
108. Hammond M D, Gale G E, Kapitan K S, Ries A, Wagner P D. Pulmonary gas exchange in humans during normobaric hypoxic exercise. J Appl Physiol (1985).
109. Torre-Bueno J R, Wagner P D, Saltzman H A, Gale G E, Moon R E. Diffusion limitation in normal humans during exercise at sea level and simulated altitude. J Appl Physiol (1985). 1985; 58:989-995.
110. Carter J, Jeukendrup A E. Validity and reliability of three commercially available breath-by-breath respiratory systems. Eur J Appl Physiol. 2002; 86:435-441.
111. Bradley A F, Severinghaus J W, Stupfel M. Effect of temperature on PCO2 and PO2 of blood in vitro. J Appl Physiol. 1956; 9:201-204.
112. Moosavi S H, Golestanian E, Binks A P, Lansing R W, Brown R, Banzett R B. Hypoxic and hypercapnic drives to breathe generate equivalent levels of air hunger in humans. J Appl Physiol (1985). 2003; 94:141-154.
113. Swenson K E, Ruoss S J, Swenson E R. The pathophysiology and dangers of silent hypoxemia in COVID-19 lung injury. Ann Am Thorac Soc. 2021; 18:1098-1105.
114. Weil J V. Variation in human ventilatory control-genetic influence on the hypoxic ventilatory response. Respir Physiol Neurobiol. 2003; 135:239-246.
115. Hodson E J, Nicholls L G, Turner P J, Llyr R, Fielding J W, Douglas G, et al. Regulation of ventilatory sensitivity and carotid body proliferation in hypoxia by the PHD2/HIF-2 pathway. J Physiol. 2016; 594:1179-1195.

### Enumerated Embodiments

Clause 1. A method of estimating pulmonary shunt and/or deadspace in a subject suffering from a pulmonary disease, comprising: (a) measuring oxygen (O2) and carbon dioxide (CO2) concentrations over multiple breaths of the subject; (b) determining a mean alveolar O2 pressure (APO2) and a mean alveolar CO2 pressure (APCO₂) based on the measurement of step (a); (c) obtaining a single arterial blood sample from the subject; (d) determining an arterial O2 pressure (aPO₂) and an arterial CO2 pressure (aPCO₂) from the arterial blood sample of step (c); (e) calculating an alveolar-arterial O2 pressure difference (AaPO₂) and an arterial-alveolar CO2 pressure difference (aAPCO₂); and (f) estimating a shunt value and/or a deadspace value using the Riley and Cournand 3-compartment model based on the AaPO₂ and aAPCO₂ of step (e).

Clause 2. A method of detecting pulmonary vascular obstruction (PVO) in a subject suffering from a pulmonary disease, comprising: (a) measuring exhaled oxygen (O2) and carbon dioxide (CO2) concentrations over multiple breaths of the subject; (b) determining a mean alveolar O2 pressure (APO2) and a mean alveolar CO2 pressure (APCO2) based on the measurement of step (a); (c) obtaining a single arterial blood sample from the subject; (d) determining an arterial O2 pressure (aPO₂) and an arterial CO2 pressure (aPCO₂) from the arterial blood sample of step (c); (e) calculating an alveolar-arterial O2 pressure difference (AaPO2) and an arterial-alveolar CO2 pressure difference (aAPCO2); (f) estimating a shunt value and/or a deadspace value using the Riley and Cournand 3-compartment model based on the AaPO2 and aAPCO₂ of step (e); and (g) detecting PVO based on the shunt value and/or deadspace value of step (f).

Clause 3. A method of determining elevated shunt and/or elevated deadspace in a subject suffering from a pulmonary disease, comprising: (a) measuring exhaled oxygen (O2) and carbon dioxide (CO2) concentrations over multiple breaths of the subject; (b) determining a mean alveolar O2 pressure (APO2) and a mean alveolar CO2 pressure (APCO2) based on the measurement of step (a); (c) obtaining a single arterial blood sample from the subject; (d) determining an arterial O2 pressure (aPO2) and an arterial CO2 pressure (aPCO2) from the arterial blood sample of step (c); (e) calculating an alveolar-arterial O2 pressure difference (AaPO2) and an arterial-alveolar CO2 pressure difference (aAPCO₂); (f) estimating a shunt value and/or a deadspace value using the Riley and Cournand 3-compartment model based on the AaPO2 and aAPCO₂ of step (e); and (g) determining that the subject has elevated shunt if the shunt value is 5% or greater and determining that the subject has elevated deadspace if the shunt value is 10% or greater.

Clause 4. A method of treating pulmonary disease in a subject in need thereof, comprising: (a) measuring exhaled oxygen (O2) and carbon dioxide (CO2) concentrations over multiple breaths of the subject; (b) determining a mean alveolar O2 pressure (APO2) and a mean alveolar CO2 pressure (APCO2) based on the measurement of step (a); (c) obtaining a single arterial blood sample from the subject; (d) determining an arterial O2 pressure (aPO2) and an arterial CO2 pressure (aPCO2) from the arterial blood sample of step (c); (e) calculating an alveolar-arterial O2 pressure difference (AaPO2) and an arterial-alveolar CO2 pressure difference (aAPCO₂); (f) estimating a shunt value and/or a deadspace value using the Riley and Cournand 3-compartment model based on the AaPO2 and aAPCO₂ of step (e); and (g) administering a treatment of the pulmonary disease to the subject based on the shunt value and/or deadspace value of step (f).

Clause 5. An apparatus for estimating pulmonary shunt and/or deadspace in a subject suffering from a pulmonary disease, comprising: (a) an oxygen (O2) analyzer configured to analyze oxygen from exhaled gas and arterial blood; (b) a carbon dioxide (CO2) analyzer configured to analyze carbon dioxide from exhaled gas and arterial blood; (c) a flow meter; and (d) a processor configured to (i) receive an oxygen input from the oxygen analyzer and a carbon dioxide input from the carbon dioxide analyzer, (ii) calculating an alveolar-arterial O2 pressure difference (AaPO2) and an arterial-alveolar CO2 pressure difference (aAPCO2) from the oxygen input and the carbon dioxide input from step (i); and estimating a shunt value and/or a deadspace value using the Riley and Cournand 3-compartment model based on the AaPO2 and aAPCO2 of step (ii).

Clause 6. The method of any one of clauses 1-4 or the apparatus of claim 5, wherein the pulmonary disease is COVID-19.

Clause 7. The method of any one of clauses 1-4, wherein measuring exhaled oxygen and carbon dioxide is performed using a flow meter.

Clause 8. The method of clause 7, wherein the exhaled oxygen and carbon dioxide are measured over 2-10 breaths of a subject.

Clause 9. The method of clause 8, wherein the subject's breathing is in a steady state.

Clause 10. The method of clause 9, wherein a tidal volume of the subjects breathing is sufficient to produce an alveolar plateau.

Clause 11. The method of any one of clauses 1-4, wherein exhaled oxygen and carbon dioxide are measured at a point halfway through an exhalation.

Clause 12. The method of clause 2, wherein detecting PVO comprises estimating a deadspace value that is greater than the deadspace of a control subject that does not have PVO.

Clause 13. The method of clause 2, wherein detecting PVO comprises detecting estimating a deadspace value that is greater than a deadspace value in a control subject that has pulmonary disease but does not have PVO.

Clause 14. The method of any one of clauses 1-4 or the apparatus of claim 5, wherein the subject is spontaneously breathing.

Clause 15. The method of any one of clauses 1-4, further comprising determining body temperature, [Hb](hemoglobin concentration), and acid-base status of the subject.

Clause 16. The method of clause 15, wherein the [Hb] and acid-base status is determined from the single arterial blood sample.

Clause 17. The method of any one of clauses 1-4, further comprising determining a cardiac output and Hb P50 (hemoglobin P50) of the subject.

Clause 18. The method of any one of clauses 1-4, wherein the cardiac output is estimated by the round number formula.

Clause 19. The method of any one of clauses 1-4, wherein the Hb P50 (hemoglobin P50) is estimated around 27 mmHg.

Clause 20. The method of any one of clauses 1, 2, or 4 further comprising determining whether the subject has elevated shunt.

Clause 21. The method of any one of clauses 20, wherein the subject has elevated shunt if the shunt value is at least about 5%.

Clause 22. The method of any one of clauses 1, 2, or 4 further comprising determining whether the subject has elevated deadspace.

Clause 23. The method of any one of clauses 22, wherein the subject has elevated deadspace if the deadspace value is at least about 10%.

Clause 24. The method of clause 22 or 23, wherein an anticoagulant is administered if the subject has elevated deadspace.

Clause 25. The method of clause 20 or 21, wherein a treatment for alveolar flooding, alveolar collapse, or pulmonary edema is administered if the subject has elevated shunt.

1. A method of estimating pulmonary shunt and/or deadspace, detecting pulmonary vascular obstruction (PVO), or determining elevated shunt and/or elevated deadspace in a subject suffering from a pulmonary disease, comprising:
(a) measuring oxygen (O₂) and carbon dioxide (CO₂) concentrations over multiple breaths of the subject;
(b) determining a mean alveolar O₂ pressure (AP_{O}_{₂}) and a mean alveolar CO₂ pressure (APCO₂) based on the measurement of step (a);
(c) obtaining a single arterial blood sample from the subject;
(d) determining an arterial O₂ pressure (aP_{O}_{₂}) and an arterial CO₂ pressure (aP_{CO}_{₂}) from the arterial blood sample of step (c);
(e) calculating an alveolar-arterial O₂ pressure difference (AaP_{O}_{₂}) and an arterial-alveolar CO₂ pressure difference (aAP_{CO}_{₂}); and
(f) estimating a shunt value and/or a deadspace value using a Riley and Cournand 3-compartment model based on the AaP_{O}_{₂} and aAP_{CO}_{₂} of step (e).

2. (canceled)

3. (canceled)

4. A method of treating pulmonary disease in a subject in need thereof, comprising:
(a) measuring exhaled oxygen (O₂) and carbon dioxide (CO₂) concentrations over multiple breaths of the subject;
(b) determining a mean alveolar O₂ pressure (AP_{O}_{₂}) and a mean alveolar CO₂ pressure (AP_{CO}_{₂}) based on the measurement of step (a);
(c) obtaining a single arterial blood sample from the subject;
(d) determining an arterial O₂ pressure (aP_{O}_{₂}) and an arterial CO₂ pressure (aP_{CO}_{₂}) from the arterial blood sample of step (c);
(e) calculating an alveolar-arterial O₂ pressure difference (AaP_{O}_{₂}) and an arterial-alveolar CO₂ pressure difference (aAP_{CO}_{₂});
(f) estimating a shunt value and/or a deadspace value using the Riley and Cournand 3-compartment model based on the AaP_{O}_{₂} and aAP_{CO}_{₂} of step (e); and
(g) administering a treatment of the pulmonary disease to the subject based on the shunt value and/or deadspace value of step (f).

5. An apparatus for estimating pulmonary shunt and/or deadspace in a subject suffering from a pulmonary disease, comprising:
(a) an oxygen (O₂) analyzer configured to analyze oxygen from exhaled gas and arterial blood;
(b) a carbon dioxide (CO₂) analyzer configured to analyze carbon dioxide from exhaled gas and arterial blood;
(c) a flow meter; and
(d) a processor configured to (i) receive an oxygen input from the oxygen analyzer and a carbon dioxide input from the carbon dioxide analyzer, (ii) calculate an alveolar-arterial O₂ pressure difference (AaP_{O}_{₂}) and an arterial-alveolar CO₂ pressure difference (aAP_{CO}_{₂}) from the oxygen input and the carbon dioxide input from step (i); and estimating a shunt value and/or a deadspace value using the Riley and Cournand 3-compartment model based on the AaP_{O}_{₂} and aAP_{CO}_{₂} of step (ii).

6. The method of claim 1, wherein the pulmonary disease is COVID-19.

7. The method of claim 1, wherein measuring exhaled oxygen and carbon dioxide is performed using a flow meter.

8. The method of claim 7, wherein the exhaled oxygen and carbon dioxide are measured over 2-10 breaths of a subject.

9. The method of claim 8, wherein the subject's breathing is in a steady state.

10. The method of claim 9, wherein a tidal volume of the subject's breathing is sufficient to produce an alveolar plateau.

11. The method of claim 1, wherein exhaled oxygen and carbon dioxide are measured at a point halfway through an exhalation.

12. The method of claim 1, wherein detecting PVO comprises estimating a deadspace value that is greater than the deadspace of a control subject that does not have PVO and/or detecting estimating a deadspace value that is greater than a deadspace value in a control subject that has pulmonary disease but does not have PVO.

13. (canceled)

14. The method of claim 1, wherein the subject is spontaneously breathing.

15. The method of claim 1, further comprising 11 determining body temperature, [Hb](hemoglobin concentration), and acid-base status of the subject and/or (2) determining a cardiac output and Hb P50 (hemoglobin P50) of the subject.

16. The method of claim 15, wherein the [Hb] and acid-base status is determined from the single arterial blood sample.

17. (canceled)

18. The method of claim 1, wherein the cardiac output is estimated by the round number formula.

19. The method of claim 1, wherein the Hb P50 (hemoglobin P50) is estimated to be around 27 mmHg.

20. The method of claim 1, further comprising determining whether the subject has elevated shunt or determining whether the subject has elevated deadspace.

21. The method of claim 20, wherein the subject has elevated shunt if the shunt value is at least about 5%.

22. (canceled)

23. The method of claim 20, wherein the subject has elevated deadspace if the deadspace value is at least about 10%.

24. The method of claim 20, wherein an anticoagulant is administered if the subject has elevated deadspace.

25. The method of claim 20, wherein a treatment for alveolar flooding, alveolar collapse, or pulmonary edema is administered if the subject has elevated shunt.
